(19) United States
(12) Patent Application Publication
(10) Pub. No.: US 2026/0290506 A1
(43) Pub. Date: Sep. 24, 2026

(54) SYSTEMS AND METHODS FOR TRAINING ANALYTE CORRECTION MODELS

(71) Applicant: Drop Bio Ltd, Kensington, NSW (AU)

(72) Inventors: Connor Patrick O'Meara, Marrickville, NSW (AU); Jim Thomas Liddle, Alexandria, NSW (AU)

(21) Appl. No.: 19/473,289

(22) PCT Filed: Jun. 26, 2024
(86) PCT No.: PCT/AU2024/050668

§ 371 Date: Oct. 7, 2025

(30) Foreign Application Priority Data
Jun. 26, 2023 (AU) 2023902008

(51) Int. Cl. G16B 40/10; G16B 40/20

(52) U.S. Cl. CPC G16B 40/10; G16B 40/20

(57) ABSTRACT

A method for generating a training set for analyte test result correction in biological samples comprising: obtaining biological samples from a plurality of donors wherein donor attribute data is collected at the same time to obtain a plurality of biological samples and associated donor attributes; dividing the collected biological samples into a plurality of aliquots and dividing the aliquots into first and second subsets of aliquots; measuring one or more analytes of the biological samples from the first subset of aliquots to provide true values of analyte characteristics for each aliquot in the first subset of aliquots; subjecting the second subset of aliquots to an environmental attribute and capturing the environmental attribute by measurement or proxy to obtain a plurality of degraded aliquot samples; for each degraded aliquot sample, measuring one or more analytes of the biological sample to provide test values of analyte characteristics; comparing the true and test values for each sample analyte to determine differences between true and respective test values; and generating a training set for a machine learning or artificial intelligence system based on relative differences between the true and test values corresponding to environmental and/or donor attributes.

### CROSS-REFERENCE TO RELATED APPLICATIONS

This application is a 371 National Phase Entry of International Patent Application No. PCT/AU2024/050668 filed on Jun. 26, 2024, which claims the benefit of Australian Provisional Patent Application No. 2023902008 filed on Jun. 26, 2023, the contents of which are incorporated herein by reference in their entirety.

### FIELD OF THE INVENTION

The present invention relates to data training sets to train a test correction system, systems and methods for collection and analysis of biological patient samples, and in particular for the correction of test result analysis of a biological patient sample to correct for the effects of delay between sample collection and analysis thereby providing quantifiable data from a sample.

### BACKGROUND OF THE INVENTION

Any discussion of the background art throughout the specification should in no way be considered as an admission that such background art is prior art, nor that such background art is widely known or forms part of the common general knowledge in the field in Australia or worldwide as at the priority date of the present application. All references, including any patents or patent applications, cited in this specification are hereby incorporated by reference, which means that it should be read and considered by the reader as part of this text.

Visiting a pathology collection center for routine blood testing is often a time consuming, disruptive, and costly process, particularly for patients living in rural areas or that require frequent care/testing. Patients are now able to self-collect many types of biological samples without the need for specialized equipment or training, which is termed “remote,” “patient-led,” or “at-home” sample collection. Remote sampling aims to maximize convenience and accessibility whilst still maintaining quality. The primary challenge associated with remote sample collection is preventing, controlling, and/or compensating for sample changes that occur during transit. Results from some blood tests remain unchanged at ambient temperatures, differing by only a few percent even after multiple days. However, the majority of tests show significant changes and potentially erroneous results after twelve to twenty-four hours held at known conditions that could lead to misdiagnoses if analyzed. In view of the clinical risks associated with erroneous test results, sample limits for quality control purposes have been established that qualitatively define if a sample is of an acceptable quality to test a given analyte. Thus, prior methods only address qualitative assessments, i.e., whether a sample is good or bad, a binary decision upon whether to analyze the sample or not. These methods mandate any sample exceeding these thresholds need to be recollected, which increases the patient burden and leads to avoiding or delaying medical intervention. These thresholds are also created under the assumption that a sample is stored at a constant room temperature, which is not suitable for dynamic processes akin to the diurnal and seasonal temperature fluctuation to which a blood sample may be subjected during transit.

The market for at-home medical testing for prevention and monitoring of disease has grown, due to its low cost, convenience, and association between early detection of health issues with improved outcomes. Consequently, there is a growing need for shipments of biological samples through normal postage or courier channels. Such shipping methods, however, are typically uncontrolled, i.e., do not offer any biological or environmental control systems to preserve the integrity of the sample during shipping before arriving at the test lab for analysis. Standard shipping methods such as national mail or delivery services such as, for example, DHL, FedEx, UPS, and the like, reduce overall costs and increase convenience. However, absent specific packaging and/or sample preparation, the samples are exposed to uncontrolled temperatures, and the temperature of each shipping day may vary with the ambient temperature between day and night or the shipping route which can typically take a few days to usually no more than about a week, where the sample environment is unknown. Due to the time delay related to shipping, most if not all samples collected by patients at-home yields results that exceed these qualitative thresholds thus preventing accessibility of testing from an at-home setting. Because of this finite stability using existing methods, the market for remote testing of samples is extremely limited.

US 2014-0342938 A1 provides a limited example of existing methods for determining whether a sample is appropriate to test. Namely, it provides a qualitative assessment of RNA quality or integrity of an RNA sample but does not teach or suggest a solution for correction of analyte measurements under dynamic environmental conditions.

Similarly, US 2019-0155987 A1 provides a methodology for determination of biological sample quality when the sample history is unknown and without requiring additional equipment. However, this solution again does not quantify or correct a test analyte from a sample, which has undergone dynamic conditions in transit from the time of collection until processing.

Self-collection and shipping of blood samples in particular requires care as blood cells naturally degrade once extracted from the body in a process known as blood lysis (hemolysis) whereby red blood cells rupture and their contents (cytoplasm) leak out into the surrounding fluid (e.g., blood plasma). Presently, this poses a significant challenge for testing of remotely collected blood samples since analysis of the sample must be obtained before the cell lysis occurs, as this can cause interferences with laboratory analyzers and directly impact the accuracy of test results. After the blood collection process, in vitro hemolysis can still occur in a sample due to external factors, such as prolonged storage, incorrect storage conditions and excessive physical forces by dropping or vigorously mixing the sample tube, or issues with improper practices or other difficulties with the initial blood collection. The risk of this occurring is heightened in an uncontrolled sample transit managed through normal postage or courier channels. While the use of environmental controls (e.g., thermally controlled packaging) reduces the deterioration of biological samples, they are more expensive and impractical when the samples are collected outside of a medical facility or where the patient lives, for example, in a rural location. There remains a significant need for testing of remotely collected samples and for low-cost shipping methods of the same, which expand the catalog of the currently very limited set of available medical tests capable of being performed on a remotely collected blood sample.

Given the sample degradation and hemolysis that can occur in at home collected samples, there is a pressing need for low-cost systems and methods of creation of a data set, capable of training a model that calculates a correction factor, to increase the number of analytes capable of being tested from a remotely collected blood sample. These embodiments provide a means for correcting measured test values obtained in the test laboratory of a degraded biological sample to more closely approximate the true value of the analytes in the biological sample, particularly of blood samples, delivered by standard shipping methods. Applicant has unexpectedly found that creation of a data training set, and use of a trained model therefrom, can improve the performance of quantification of a biological sample by magnitudes of improvement over prior methodologies, thus allowing for many more analytes to be tested than were heretofore unable to be tested due to degradation issues beyond even a few hours of transit. This greatly reduces the need for sample recollection, by extending the quality control threshold, and such improvements then yield quantitative determinations for numerous analytes. These and other methods are detailed in the embodiments herein.

### SUMMARY OF THE INVENTION

Applicant, as detailed herein, provides a solution greatly increasing the number of analytes that can be quantified through creation of a data training set and a model trained therefrom to allow for quantitative adjustment of test results of analytes from remotely collected blood samples, thereby extending the sample quality control threshold, and reducing the requirement for sample recollection. In some embodiments, models are developed for making analyte corrections, and wherein the models include a plurality of models, systems of models, competing models, and/or ensembles of models that work together to provide a predicted analyte correction value for a given sample and a given analyte.

In a preferred embodiment Applicant details the creation of data sets and models for determining concentrations of a desired analyte from a biological sample, particularly towards whole blood samples, which are exposed to dynamic time and temperature variables, but which are captured directly or by a proxy, and wherein the dynamic variables can be fed into the models to predict a correction value for a given analyte based upon those variables, and/or donor variables, and/or degradation variables.

In a preferred embodiment, a method for generating a training set for correction of analyte test results in a biological sample, the method comprising: (a) obtaining a biological sample from a plurality of donors wherein donor attribute data is collected at the time of collecting the biological sample to obtain a plurality of biological samples and associated donor attributes; (b) dividing the collected biological sample into a plurality of aliquots and dividing the aliquots into a first subset of aliquots and a second subset of aliquots; (c) measuring one or more analytes of the biological sample from the plurality of aliquots of the first subset of aliquots to provide a true value of one or more analyte characteristics for each aliquot in the first subset of aliquots; (d) subjecting each of the plurality of aliquots of the second subset of aliquots to an environmental attribute and capturing the environmental attribute by measurement or a proxy to obtain a plurality of degraded aliquot samples; (e) for each degraded aliquot sample, measuring one or more analytes of the biological sample to provide a test value of the one or more analyte characteristics; (f) comparing the true values and test values for each of the sample analytes to determine a difference between the true values and respective test values; and (g) generating a training set for training a machine learning or artificial intelligence system, the training set based on the relative difference between the true values and the test values corresponding to the environmental attribute and/or the donor attributes.

In a further embodiment, the method further comprising obtaining a sample degradation attribute for each second aliquot.

In a further embodiment, the method wherein the donor attributes, environmental attributes, and/or degradation attributes are a proxy for such attributes.

In a further embodiment, the method further comprising quantifying a plurality of analytes of each aliquot sample to obtain a true value for each of the plurality of analytes measured.

In a further embodiment, the method further comprising wherein the second subset of aliquots is subjected to environmental conditions and capturing said environmental conditions wherein the environmental conditions are time, temperature, or both. In a further embodiment, the method wherein the environmental conditions are dynamic environmental conditions.

In a further embodiment, the method further comprising comparing the difference in the true value and the test value to construct a machine learning or artificial intelligence model capable of predicting a correction factor in order to correct test results using the machine learning or artificial intelligence model trained on the training set.

In a further embodiment, the method wherein the machine learning or artificial intelligence model is a parametric or nonparametric machine learning model; and/or wherein the machine learning or artificial intelligence model is constructed using a multivariate adaptive regression spline (MARS) method for each analyte of the biological sample of interest; and/or wherein the machine learning or artificial intelligence model is calculated using either a locally weighted scatterplot smoother (LOWESS) or a polynomial fit model for each analyte of the biological sample of interest.

In a further embodiment, the method wherein the biological sample is a blood sample and the measuring comprises measuring one or more analytes from a plasma or serum fraction of the blood sample.

In a further embodiment, the method wherein the donor attributes are selected from the group consisting of: age, sex, ethnicity, BMI, fasting status, time of day, medication, medical conditions, and combinations thereof.

In a further embodiment, the method wherein the environmental attributes comprise one or more of a proxy of time, temperature, humidity, pressure, geolocation, acceleration, and light.

In a further embodiment, the method wherein degradation attributes comprise one or more of a proxy of entropy of a biological sample, red blood cell lysis, white blood cell lysis, presence or absence of DNA, sodium, chloride, potassium, an analyte different from an analyte of interest, and evaporation. In a further embodiment, the method wherein the degradation attribute is calculated by using a first model and comprising using the calculations from the first model as inputs to a second model wherein the inputs to the second model are representative of estimated degradation of the biological sample.

In a preferred embodiment, a computer-implemented method of training a model for analyte correction comprising: (a) collecting a set of biological samples; (b) separating the set of biological samples into at least a first set of aliquots and a second set of aliquots; (c) applying one or more environmental attributes to the second set of aliquots; (d) creating a first training set comprising the first set of aliquots of biological samples, each said biological sample having a known analyte value; (e) training the model in a first stage using the first training set; (f) creating a second training set for a second stage of training comprising the second set of aliquots; (g) training the model in a second stage using the second training set; and (h) comparing the model of the first stage to the model of the second stage to define the analyte correction model.

In a further embodiment, the computer-implemented method further comprising associating a donor attribute to each of the first set of aliquots and the second set of aliquots. In a further embodiment, the computer-implemented method further comprising associating a degradation attribute to the biological sample in each of the second set of aliquots.

In a further embodiment, the computer-implemented method wherein the environmental attribute and/or the degradation attribute is obtained by a proxy.

In a further embodiment, the computer-implemented method further comprising a third training set, said third training set defined by using the analyte correction model to predict a degradation attribute, and training a stacked model based upon the third training set.

In a preferred embodiment, a method of creating a model for analyte stability comprising: (a) collecting a set of test value measurements for a given analyte, each test value measurement having a corresponding true value measurement wherein the given analyte is obtained from a mammalian biological sample; (b) associating each test value measurement and corresponding true value measurement with a donor attribute and storing the test value measurements and true value measurements in a database; (c) associating an environmental attribute to each test value measurement to create a modified set of test values; (d) creating a first training set comprising the collected set of test values, the corresponding true values, and the modified set of test values; and (e) training a sample stability model with the first training set.

In a preferred embodiment, a method of building a computer implemented model to correct results obtained from a remotely collected biological sample, the method comprising: (a) obtaining a biological sample from a plurality of donors and dividing each biological sample into a plurality of test aliquots and at least one control aliquot; (b) for at least one analyte in the control aliquot, obtaining a true value result and storing the true value result in a database; (c) subjecting each test aliquot to a test time, a test temperature, or both; (d) thereafter and from each test aliquot, obtaining test value results for the at least one analyte and storing the test value results in the database; (e) retrieving the true value result and the test value results from the database and determining a difference between the true value result and each test value result; and (f) using the true value result, the test value results, and the determined differences together with corresponding test times and/or test temperatures to train a stability consensus machine learning model to predict correction coefficients for correcting analyte measurement results obtained from a remotely collected biological sample, the predicted correction coefficients to correct for analyte drift due to time and/or temperature changes that occurred during transit of the remotely collected biological sample.

In a preferred embodiment, a method of correcting test results of patient biological samples having experienced a delay in the time between collection and analysis of the patient biological sample, the method comprising: (a) providing a collection container for receiving patient biological samples received in a packaged state with sample packaging comprising: (i) one or more environmental sensors and a data logger, or proxies thereof, configured to measure and store environmental attribute data to which the patient biological sample was exposed during transit; and (ii) patient attribute data with respect to attributes of the patient; (b) providing a data reader for downloading the environmental attribute data from the data logger, or use of historical, weather, and/or shipment tracking information as a proxy for environmental attribute data, and associating the environmental attribute data and patient attribute data with the patient biological sample; (c) providing biological sample test hardware for measuring one or more analyte test data of the patient biological sample; (d) providing degradation attribute hardware for determining one or more degradation attributes of the patient biological sample; (e) providing a database for storing the patient attribute data comprising, with respect to the biological sample: (i) analyte test data; (ii) environmental attributes; (iii) degradation attributes; and (iv) donor attributes; (f) providing a processor configured to receive the patient attribute data from the database and, using a machine learning model, generate a corrected value of the analyte test data; and (g) providing an output module for outputting the corrected analyte test values, the corrected analyte test values being indicative of a true analyte measurement of an undegraded patient biological sample.

In a further embodiment, the method wherein weight is applied to a variable within the model wherein the weight is based upon the known environmental attribute associated with a sample. In a further embodiment, the method wherein the weight is determined by one or more of: filter methods based on target variable, variance, information gain, mutual information, and chi-squared tests; wrapper methods; and embedded methods that combine feature selection with model building process.

In a further embodiment, the method wherein the environmental attribute is time, temperature, humidity, pressure, geolocation, acceleration, light, or combinations thereof.

In a further embodiment, the method wherein the donor attribute is selected from the group consisting of: age, sex, ethnicity, BMI, fasting status, time of day, medication, medical conditions, and combinations thereof.

In a further embodiment, the method wherein the degradation attribute is selected from the group consisting of: DNA, white blood cells, hemoglobin, sodium, chloride, potassium, a color, one or more of a proxy of entropy of a biological sample, red blood cell lysis, presence or absence of DNA, an analyte different from an analyte of interest, evaporation, and combinations thereof.

In a further embodiment, the method wherein the model is determined by finding an optimal value for a parameter in order to minimize an error or loss function by selecting from a gradient descent model, close-form solution, regularization, Bayesian inference estimates, and/or cross-validation.

In a further embodiment, the method wherein the second subset of aliquots is exposed to known dynamic environmental conditions, wherein said known dynamic environmental conditions are recorded as time and temperature, and wherein the conditions of temperature can be obtained by a proxy corresponding to an ambient temperature of the second aliquot.

In a further embodiment, the method wherein donor information is obtained from an electronic medical record.

In a further embodiment, the method wherein the degradation attributes from the biological sample are provided by the attributes within the biological sample itself.

In a preferred embodiment, a training set generated by the method.

In a preferred embodiment, a value correction model for correcting analyte test values obtained from a degraded biological sample, the value correction model comprising: a machine learning or artificial intelligence model trained on a training set, the training set comprising difference data between analyte measurements from a degraded biological sample and a true value of an undegraded biological sample; an input module adapted to receive a plurality, a proxy, or covariate of test analyte values from a degraded aliquot of a biological sample; environmental attribute data in respect of environmental attributes to which the degraded aliquot has been exposed; an analysis processor to predict a corrected analyte measurement from the test analyte values on the basis of a model trained on a training set comprising relative difference data between true analyte values and test analyte values from a degraded biological sample; and an output module adapted to generate a corrected analyte value indicative of the true analyte value of the biological sample without degradation.

In a further embodiment, the value correction model further comprising a degradation attribute data in respect of degradation attributes to which the degraded aliquot has undergone.

In a further embodiment, the value correction model further comprising a donor attribute data in respect to attributes of the donor.

In a preferred embodiment, a system for correcting test results of donor biological samples having a known environmental attribute, the system comprising: a collection container for receiving a donor biological sample, said donor biological sample having experienced a delay in analyte analysis wherein the delay comprises a sufficient time for an environmental attribute to cause a change in the donor biological sample; biological sample test hardware for performing one or more analyte test data of the donor biological sample; degradation attribute hardware for determining one or more degradation attributes of the donor biological sample; a database for storing donor data comprising, with respect to the donor biological sample: analyte test data, environmental attributes, degradation attributes, and donor attributes; a processor configured to receive the donor data from the database and, using a machine learning or artificial intelligence model, generate a corrected value of the analyte test data; and an output module for outputting corrected analyte test values, the corrected analyte test values being indicative of a true analyte measurement of an undegraded donor biological sample.

In a further embodiment, the system wherein the donor biological sample is received in a packaged state with sample packaging comprising: one or more environmental sensors and a data logger or proxies thereof configured to measure and store environmental attribute data to which the donor biological sample was exposed during transit; and donor attribute data with respect to attributes of the donor.

In a further embodiment, the system further comprising a data reader for downloading the environmental attribute data from the data logger and associating the environmental attribute data and donor attribute data with the donor biological sample.

In a further embodiment, the system wherein the machine learning model is trained on a training set comprising relative difference data between true analyte values and test analyte values from a degraded biological sample discordant from the expected.

In a further embodiment, the system further comprising a client-accessible server comprising: a processor configured for performing computer program code instructions; memory for storing computer code instructions for execution by the processor; a database for storage of a value correction model for correction of analyte measurements of degraded biological samples; and a user interface accessible by a client device for: receiving measurement test data and associated degradation attribute data from a client and outputting corrected true value measurement data to the client device.

In a preferred embodiment, a computer program product for generating a model for an analyte of a biological sample for correction of analyte test values obtained from a degraded biological sample, the computer program product comprising a nontransitory computer-readable storage medium having computer-readable program code portions stored therein, the computer-readable program code portions comprising: a first portion configured to provide a machine learning model trained on a training set, the training set comprising difference data between analyte measurements from a degraded biological sample and an undegraded biological sample; a second portion configured to provide for downloading environmental attribute data from a data logger included with a patient biological sample exposed to dynamic environmental conditions and associating the environmental attribute data with the patient biological sample; a third portion configured to provide for measuring one or more analyte test data of the patient biological sample; a fourth portion configured to provide for determining one or more degradation attributes of the patient biological sample; a fifth portion configured to provide for determining and/or recording patient data comprising, with respect to the biological sample: analyte test data, environmental attributes, degradation attributes, and patient attributes, and recording the patient data; a sixth portion configured to provide associating the patient data with the patient biological sample; a seventh portion configured to provide for generating a corrected analyte value from the patient data using the machine learning model; and an eighth portion configured to provide for outputting corrected analyte test values, the corrected analyte test values being indicative of a true analyte measurement of an undegraded patient biological sample.

In a preferred embodiment, a system for predicting a correction coefficient for remotely collected biological samples, the system comprising: (a) a computer comprising a processor that processes machine-readable instructions and a memory that stores the machine-readable instructions which can be executed by a process configured to the computer; (b) an algorithm to predict correction coefficients for analyte measurements taken from a biological sample that was collected at a remote location and subjected to at least a delay in analysis, the correction coefficients, when applied, to adjust the analyte measurement from the remotely collected biological sample to an adjusted measurement value within an acceptable degree of error of a true measurement value; (c) a database storing training data, the training data comprising a set of analyte control values and a set of analyte test values wherein each analyte test value is associated with an environmental attribute of time, temperature, or both; and (d) wherein when executed, the instructions cause the processor to: (i) retrieve the training data from the database; (ii) determine a difference between each analyte control value in the set and one or more test values for each corresponding analyte; (iii) add the difference values to the training data; (iv) input the training data, including the differences, into the database to build a relationship between the analyte control value and the analyte test value and define a difference between the analyte control value and the analyte test value in relation to the environmental attributes; and (v) weight test times and/or test temperature for predicting a correction coefficient that corrects a test value for analyte drift from the control value.

### BRIEF DESCRIPTION OF THE FIGURES

FIG. 1 shows a flowchart related to the creation of a stability model training data set using blood samples incorporating patient health conditions and subjected to environmental and conditions experienced during transit.

FIG. 2 shows a method of creation of a stability model validation set using donor matched blood samples and demonstrated in a trial under real-world conditions where the donor samples were posted to the analyzing laboratory with environmental attributes measured by a data logger.

FIG. 3A depicts the improvement of an analyte calculation from a blood sample, when defining time as a variable in the analysis, with FIG. 3B showing the absolute percent improvement compared to an uncorrected sample.

FIGS. 4A and 4B depict a model of sample stability that accommodates time and a fixed temperature, which improves accuracy for samples transported in a controlled and uncontrolled environment.

FIGS. 5A, 5B, 5C, and 5D depict a plot of dynamic temperature fluctuation from diurnal and seasonal changes collected by temperature data loggers from real-world biological samples shipped to the laboratory by mail.

FIGS. 6A and 6B depict a model of sample stability that accommodates variable temperature, which improves accuracy for samples transported in a controlled and uncontrolled environment.

FIG. 7A-7F relate to seasonal temperature variance from data loggers or by a proxy, with FIGS. 7A, 7B, 7C, and 7D comparing the temperature during transport of a blood sample across the four seasons and comparing that to an actual or measured temperature with an estimated temperature, by obtaining regular temperature information for the location of the sample at a given time. FIGS. 7E and 7F then depict a plot comparing the model improvements using either the measured temperature, via a data logger (real) or the predicted condition of the sample period (mock) using temperature estimates.

FIGS. 8A and 8B depicts a model of sample stability that accommodates patient demographic information (e.g., sex, age, and ethnicity) improves test accuracy for samples transported in uncontrolled environments.

FIGS. 9A and 9B depicts a model of sample stability that accommodates direct, indirect, and predicted sample degradation information improves test accuracy for samples transported in uncontrolled environments.

FIGS. 10A and 10B depict a model of sample stability that accommodates patient health information (e.g., body mass index [BMI], medication, and medical conditions) improves test accuracy for samples transported in uncontrolled environments.

FIGS. 11A and 11B depict a model of sample stability that applies variable weights to different data points improves test accuracy for samples transported under specific use cases (e.g., sample transported in an uncontrolled environment and received by the laboratory within one day of collection). This figure depicts a bar plot showing the % error (FIG. 11A) and % accuracy improvement (FIG. 11B) over 1 day associated with uncorrected test results as the control, test results corrected using a model trained on evenly weighted data and test results corrected using a model trained on data heavily weighted for To accuracy for an analyte.

FIGS. 12A and 12B depict flowcharts, with FIG. 12A being a flowchart related to the creation of a degradation model training data set using blood samples subjected to environmental conditions experienced during transit and FIG. 12B being a method of creation of a validation set consisting of common sample collection and storage mistakes used for testing degradation models.

FIG. 13 shows a method of creation of a degradation model validation set using donor matched blood samples and demonstrated in a trial under real-world conditions where the donor samples were posted to the analyzing laboratory.

FIGS. 14A and 14B depict a model that applies an assessment using a degradation model where samples with an observed sample quality significantly discordant from the expect quality are removed improves test accuracy for samples transported in uncontrolled environments. This figure depicts a bar plot showing the % error (FIG. 14A) and % accuracy improvement (FIG. 14B) associated with uncorrected test results as the control, test results corrected using a variable temperature (FIG. 6), and test results corrected using a variable temperature plus removal if expected sample quality exceeds observed sample quality for an analyte.

FIGS. 15A and 15B depict a sample stability model showing the incremental improvement in test accuracy for samples transported in both controlled and uncontrolled environments after the combination of multiple unexpected findings.

FIG. 16 shows a flowchart related to the data structure of the training set, the prepossessing, model training, selection and performance assessment of stability and degradation models.

FIG. 17 shows a computing device on which the various embodiments described herein may be implemented in accordance with an embodiment of the present invention.

FIG. 18 shows a client-server implementation of a test correction platform for correction of test data from a client device by a client accessible server device providing a software as a service application.

### DETAILED DESCRIPTION OF THE INVENTION

Those skilled in the art will appreciate that the invention described herein is susceptible to variations and modifications other than those specifically described. The invention includes all such variation and modifications. The embodiments herein comprise one or more of the steps features, formulations, and compounds referred to or indicated in the specification, individually or collectively and any and all combinations of the steps or features. Although any methods and materials similar or equivalent to those described herein can be used in the practice or testing of the present invention, preferred methods and materials are described. It will be appreciated that the methods, apparatus, and systems described herein may be implemented in a variety of ways and for a variety of purposes. The description here is by way of example only.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, the following terms are defined below.

The term “test value” refers to the raw output measurement from an analyzer for an analyte in a biological sample that is uncorrected.

The term “true value” refers to a measurement of an analyte in a biological sample shortly after sample collection, or with minimal delay, and is considered to be the correct value of the analyte measurement with respect to best practice measurement methods.

The term “corrected value” refers to a test value which has been corrected according to a correction factor or equation derived from the machine learning model disclosed herein to obtain a measurement value as close as possible to the true value.

The term “proxy” refers to any covariate used as a substitute for an independent variable based on an association and/or correlation with the independent variable discussed herein. By way of example, quantification of plasma color change by light absorbance is a proxy for plasma hemoglobin concentration.

The articles “a” and “an” are used herein to refer to one or to more than one (i.e., to at least one) of the grammatical object of the article. By way of example, “an element” refers to one element or more than one element.

The term “about” is used herein to refer to quantities that vary by as much as 10% to a reference quantity. The use of the word “about” to qualify a number is merely an express indication that the number is not to be construed as a precise value.

The term “model” means a mathematical representation of a process that is trained on data to make predictions or decisions.

The phrase “and/or” as used herein in the specification and in the claims should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The various methods or processes outlined herein may be coded as software that is executable on one or more processors that employ any one of a variety of operating systems or platforms. Additionally, such software may be written using any of a number of suitable programming languages and/or programming or scripting tools, machine learning or AI libraries, and also may be compiled as executable machine language code or intermediate code that is executed on a framework or virtual machine. In this respect, various inventive concepts may be embodied as a computer readable storage medium of any form or other nontransitory medium or tangible computer storage medium encoded with one or more programs that, when executed on one or more computers or other processors, perform methods that implement the various embodiments of the invention discussed above. The computer readable medium or media can be transportable, or accessed remotely, such as via a cloud server, or Web site, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer, multiple separate computers, or other processor to implement various aspects of embodiments as discussed above. Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. For the purpose of this specification, where method steps are described in sequence, the sequence does not necessarily mean that the steps are to be carried out in chronological order in that sequence, unless there is no other logical manner of interpreting the sequence.

Measurement of analytes (also referred to as tests) from a blood sample, is a routine medical procedure that allows for a medical practitioner to evaluate a patient for certain issues. Analytes here are broadly referring to a measurement of some element from the biological sample. In many cases, blood samples are collected at a hospital or outpatient office and the samples are immediately analyzed or are manipulated in certain manners, such as separating the plasma from the hematocrit, and/or freezing a sample after such separation, or preserving the sample in other known methodologies that prevents degradation of a sample. Even under such care, sampling error rates are approximated at between 0.1% and 6.5%. However, the largest issue is patient noncompliance, meaning that the test is not even taken, which is estimated at between 6.8% and 62%. Indeed, a lack of access to routine blood testing impacts around 43% of the world's population that are classified as living in a rural or remote area. This limited access to health services leads patients to delay or avoid essential medical intervention and participation and adherence in clinical trials, while also driving life expectancy gaps across age, sex, and socioeconomic statuses. Therefore, remote testing is likely to improve patient health and compliance with testing, as the burden for such testing will be reduced. However, current protocols make remote sample collection all but impossible for virtually all desired analytes, thus preventing its widespread use.

Self-collection of biological samples has the potential to significantly improve outcomes for long-term monitoring of patient disease state or progression. The decentralized nature of remote sample collection, which enables a patient to collect their own blood samples in the comfort of their home for shipping to the test lab via normal post or courier services, provides significant advantages in monitoring for the presence or progression of a disease state in a patient. Especially for nonurgent preventative care, such as periodic blood testing panels, shipment by the patient of self-collected biological samples via standard shipment method encourages the patient to submit biological samples for testing earlier and more frequently. Such early and frequent testing has the potential to detect health issues earlier in the course of the disease, when alterations in lifestyle or medical care can significantly lessen the negative health effects of those issues (the early detection of cancer is one example where frequent and early blood testing increases the likelihood of complete recovery from the disease). As many diseases develop over time, such early and frequent testing has the potential to identify health issues early in the course of the disease.

In the current state of the art, remote testing of biological samples, and specifically those of whole blood samples is limited to a few analytes based on their stability, thus limiting remote collection's commercial usefulness and scale, despite significant desire by the population for such solutions. While hospital or laboratory sample collection can rapidly process a sample to be analyzed in a timely manner after collection, current technology for remote collection of samples makes such professional sample preparation and proximate time of processing to the collection time, all but impossible. A remotely collected sample here preferably means a sample that is whole blood, but may optionally include a stabilizer, such as heparin. Typically, these remotely collected samples are not spun and separated into plasma, or otherwise frozen immediately after collection, but in all cases, such remotely collected samples are not able to be immediately analyzed after their collection or separated and stabilized according to good lab practices to maintain analyte stability for subsequent analysis. Therefore, analytes within the blood will begin to drift from their true value due to various factors. For example, in a remotely collected sample, cell lysis occurs after at least several hours, and can cause interferences with laboratory analyzers and directly impact the accuracy of test results. After the blood collection process, in vitro hemolysis can still occur in a sample due to external factors, such as prolonged storage, incorrect storage conditions and excessive physical forces by dropping or vigorously mixing the tube, or issues with improper practices or other difficulties with the initial blood collection. The risk of this occurring is heightened in an uncontrolled sample transit managed through normal postage or courier channels. Indeed, for this reason, analytes are presently unable to be tested from a remotely collected sample, after having been stored for just a few hours in an uncontrolled environment, because existing platforms for their testing show error rates that are unacceptable.

The main challenge associated with remote sampling is therefore preventing, controlling, and/or compensating for sample changes that occur during transport. Prevention or controlling, require specialized shipment protocols or sample preparation that are unsuitable for remote sampling. Incidentally, not all tests drift away from the true value at the same rate, and not all drift in the same direction. Thus, while one analyte may be stable for forty-eight hours, another may be stable for only twelve hours before its value drifts down, and yet another may be stable for only four hours before its value drifts up. Indeed, when there will be a known delay in testing of a sample, the prior art teaches that a qualitative analysis, such as it has been at least twelve hours since the sample has been collected, is used to determine if the sample is of sufficient quality for testing the given analyte, not as to its quantified value.

Applicant provides an elegant technical solution to this problem. Applicant's technical solution provides for an improved accuracy, improved precision, reduced sample recollection, and reduction of error rates for numerous analytes, such that the number of tests able to be processed by remote collection is dramatically increased. Furthermore, errors among all analytes are reduced through creation of a data training set and model to provide corrected values for a given analyte. Thus, by use of the technological solution provided herein, a delay between blood sample collection and analysis can be processed by a model to dramatically increase accuracy, precision, and increase the number of analytes that can be quantified instead of requiring a recollection of the sample. The embodiments herein increase the accuracy of the quantified analyte as compared to prior art solutions, and increase precision, allowing for repeatability of testing irrespective of the time and storage conditions for remotely collected blood samples.

Table 1 contrasts the current state of the art to the invention for processing at-home collected samples. The current state of the art is defined by the guidelines that clinical pathology follows to achieve a specified level of accuracy. These guidelines mandate that a whole blood sample, if stored at room temperature (22° C.), must be processed into serum/plasma within the recommended time frame of twelve to twenty-four hours of collection. Due to the extended time delay and variable temperatures associated with uncontrolled shipment of a sample by mail, 100% of at-home collected samples will be rejected from analysis using current methods as no sample meets the recommended time and temperature storage requirement. However, with the application of the invention described herein, Applicant has established a new threshold for processing whole blood samples and/or deriving a final analyte value that extends the reach of existing tests and unlocks new test capabilities for analysis of samples collected at-home. Applicant has not only increased the maximum time delay in processing of whole blood to plasma/serum by up to 6.5 days, but has also increased the maximum storage temperature up to 37° C. Using these new thresholds Applicant has reduced the rate of sample rejection for at-home collected samples to single digits for eighty-seven common test analytes.

TABLE 1
ANALYTES SUITABLE FOR TESTING WITH
INVENTION COMPARED TO THE PRIOR ART

| | Existing | | New | |
|---|---|---|---|---|
| Existing | Rejection | New | Rejection | |
| Threshold | Rate | Threshold | Rate | Test Analyte |
| <24 hours @ | 100% | up to 3 days | 3% | ba-count, ba-percent, eo-count, |
| 22° C. | | @ 37° C. | | eo-percent, hct, hgb, ly-count, ly- |
| | | | | percent, mch, mchc, mcv, mo- |
| | | | | count, mo-percent, mpv, ne-count, |
| | | | | ne-percent, plt, rbc, rdw, rdwsd, |
| | | | | wbc, active_peptide_yy, ca125, |
| | | | | ca15-3, ca19-9, cea, ghrelin_total, |
| | | | | glp1_active, glucagon, insulin, |
| | | | | pancreatic_polypeptide_f, psa-total |
| <12 hours @ | 100% | up to 4 days | 2% | alp, apo-al, hba1c |
| 22° C. | | @ 37° C. | | |
| <24 hours @ | 100% | up to 4 days | 2% | adipsin, afp, bdnf, chemerin, |
| 22° C. | | @ 37° C. | | cortisol, cpeptide, eotaxin3, |
| | | | | estradiol, fgf23, gdf15, hgf, |
| | | | | icam1, ifng, il10, il17a, il1b, il1ra, |
| | | | | il2ra, il4, il6, il6ra, kim1, leptin, |
| | | | | mcp1, ngal, opn, pai1, progesterone, |
| | | | | rankl, shbg, st2, t3, t4, |
| | | | | testosterone, tnfa, albumin |
| <12 hours @ | 100% | up to 5 days | 1% | bilirubin-direct, bilirubin-total, |
| 22° C. | | @ 37° C. | | creatinine, ggt |
| <12 hours @ | 100% | up to 7 days | 1% | alt, ast, bicarbonate, calcium, |
| 22° C. | | @ 37° C. | | cholesterol, crp, glucose, hdl, ldl, |
| | | | | protein-total, triglycerides, urea |

Table 1 provides a dramatic demonstration of the current remote blood sampling issues in the columns “Existing Threshold” and “Existing Rejection Rate,” namely that virtually all samples collected in an at-home setting and transported by mail are rejected or must be recollected after just a few hours if samples are assessed using the current clinical guidelines for acceptance of blood samples for analysis. By contrast, the embodiments of the invention described herein shown in the columns “New Threshold” and “New Rejection Rate” yield multiple days of improvement in transportation latency between the time of blood sample collection and the time of processing and are still able to obtain an accurately quantified result of a given analyte from that sample and dramatically lowering rejection rates.

Table 2 provides a list of eighty-seven analytes, with full and abbreviated names, where improvements in transportation latency between the blood sample collection and the processing, and testing accuracy were made. Analytes include the most common clinical and research test panels, such as comprehensive metabolic panel, lipids, electrolytes, proteins, liver, kidney and thyroid function, full/complete blood counts, hormones, cancer antigens, cardiac and immune biomarkers, and many others. These tests were chosen as they represent a cross section of different organ functions, metabolites, proteins, and other small molecule sources that can be analyzed on a wide variety of common industry equipment including biochemical and immunoassay analyzers. Due to the successful improvement in test accuracy seen across all categories, the Applicant expects a similar performance for tests not described herein.

TABLE 2
DEFINITION OF ABBREVIATED TEST NAMES

| Abbreviated Test | | Abbreviated | |
|---|---|---|---|
| Name | Full Test Name | Test Name | Full Test Name |
| active-peptide-yy | * | il17a | Interleukin 17a |
| adipsin | * | il1b | Interleukin 1 beta |
| afp | Alpha fetoprotein | il1ra | Interleukin 1 receptor |
| | | | antagonist |
| albumin | * | il2ra | Interleukin 2 receptor |
| | | | subunit alpha |
| alp | Alkaline Phosphatase | il4 | Interleukin 4 |
| alt | Alanine aminotransferase | il6 | Interleukin 6 |
| apo-a1 | Apolipoprotein A1 | il6ra | Interleukin 6 receptor |
| ast | Aspartate aminotransferase | insulin | * |
| ba-count | Basophil-count | kim1 | Kidney injury molecule 1 |
| ba-percent | Basophil-% | ldl | Low-density lipoprotein |
| bdnf | Brain-derived | leptin | * |
| | neurotrophic factor | | |
| bicarbonate | * | ly-count | Lymphocyte-count |
| bilirubin-direct | * | ly-percent | Lymphocyte-% |
| bilirubin-total | * | mch | Mean corpuscular hemoglobin |
| ca125 | Cancer antigen 125 | mchc | Mean corpuscular hemoglobin |
| | | | concentration |
| ca15-3 | Cancer antigen 15-3 | mcp1 | Monocyte chemoattractant |
| | | | Pprotein 1 |
| ca19-9 | Cancer antigen 19-9 | mcv | Mean corpuscular volume |
| calcium | * | mo-count | Monocyte-count |
| cea | Carcinoembryonic | mo-percent | Monocyte-% |
| | antigen | | |
| chemerin | * | mpv | Mean platelet volume |
| cholesterol | * | ne-count | Neutrophil-count |
| cortisol | * | ne-percent | Neutrophil-% |
| cpeptide | * | ngal | Neutrophil gelatinase- |
| | | | associated lipocalin |
| creatinine | * | opn | Osteopontin |
| crp | C-reactive protein | pai1 | Plasminogen activator |
| | | | inhibitor 1 |
| eo-count | Eosinophil-count | pancreatic- | * |
| | | polypeptide-f | |
| eo-percent | Eosinophil-% | plt | Platelet |
| eotaxin3 | * | progesterone | * |
| estradiol | * | protein-total | * |
| fgf23 | Fibroblast growth factor 23 | psa-total | Prostate specific antigen |
| gdf15 | Growth differentiation | rankl | Receptor activator of |
| | factor 15 | | nuclear factor-κB ligand |
| ggt | Gamma glutamyl transferase | rbc | Red blood cell |
| ghrelin-total | * | rdw | Red cell distribution width |
| glp1-active | Glucagon-like peptide 1 | rdwsd | Red cell distribution width |
| | | | standard deviation |
| glucagon | * | shbg | Sex hormone binding |
| | | | globulin |
| glucose | * | st2 | Suppression of |
| | | | tumorigenicity 2 |
| hba1c | Hemoglobin A1C | t3 | Triiodothyronine |
| hct | Hematocrit | t4 | Thyroxine |
| hdl | High-density lipoprotein | testosterone | * |
| hgb | Hemoglobin | tnfa | Tumor necrosis factor alpha |
| hgf | Hepatic growth factor | triglycerides | * |
| icam1 | Intercellular adhesion | urea | * |
| | molecule-1 | | |
| ifng | Interferon gamma | wbc | White blood cell |
| il10 | Interleukin 10 | | |

* = Abbreviated Test Name and Full Test Name are identical

The samples obtained by remote collection, by their very nature, are degraded. As used herein, a degraded sample refers to a biological sample which has experienced a delay between the time of sample collection and time of analysis, during which the sample will typically undergo changes which affect the test value of an analyte once the sample is analyzed most remotely collected samples will also be degraded samples. Such changes in sample analyte concentration are often dynamic, where a balance between analyte accumulation from cell lysis and enzymatic/metabolic breakdown compete to influence overall sample changes. Certain analytes measured via blood tests remain unchanged in blood at room temperature and only change by a few percent even after several days. However, for many analytes any delay in sample processing leads to potentially erroneous results due to degradation. Existing methods have been only used to establish sampling thresholds for quality control purposes and, to estimate shelf life. However, Applicant has identified gross errors in the existing stability models and therefore such existing models are not suitable for dynamic processes such as those subject to daily and seasonal temperature fluctuations that a blood sample may be exposed to during transport. Indeed, this explains why current protocols do not allow for most analytes to be quantified in this manner due to the significant rates of error. Thus, Applicant has created a new method and process to quantify biological analytes with increasing accuracy that was heretofore impossible, and thus has increased the accuracy of quantified data from a degraded sample.

### Creation of Training and Validation Data Sets

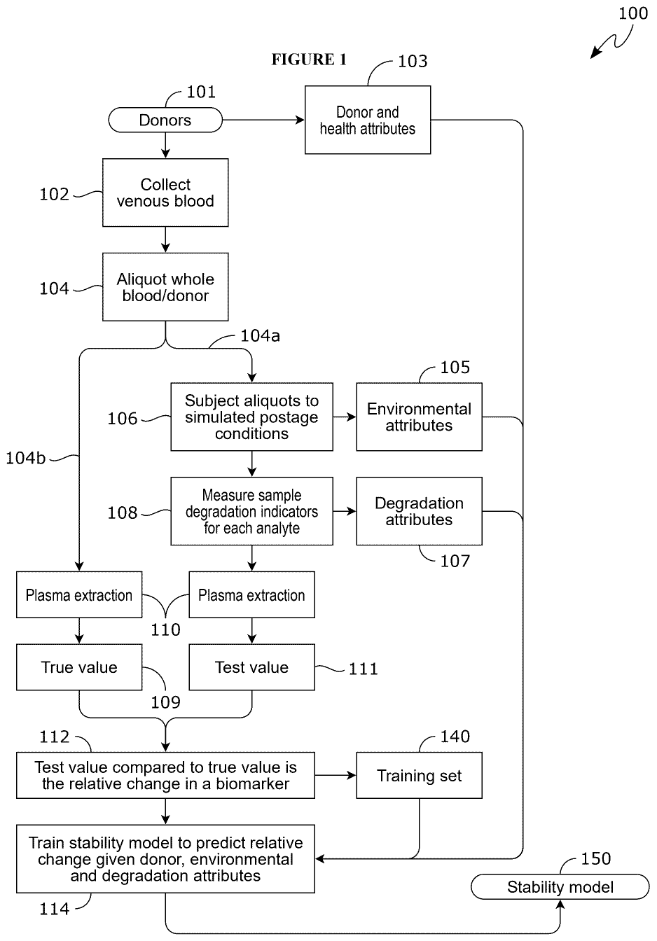

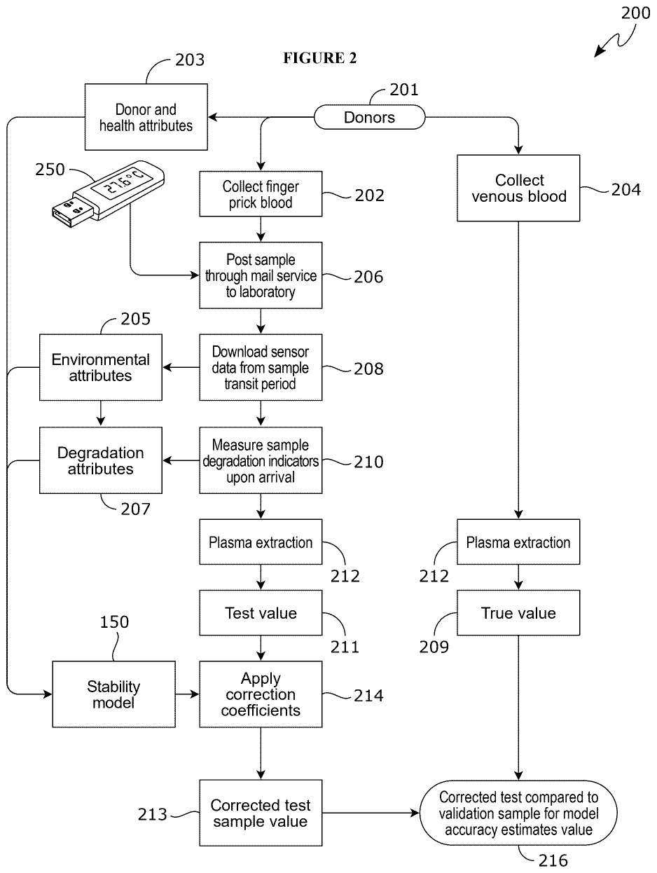

The stability model of the present disclosure is predicated upon an experimental design sufficient to obtain a quantity of data, with sufficient variety to provide meaningful training to the model via a data training set. FIGS. 1 and 2 depict flowcharts of the creation of a data training set and then a validation training set as detailed herein. To create the data sets for the machine learning models described herein a study method (100) (FIG. 1) was designed to obtain a blood sample from each of the trial participants to undertake onsite testing of the blood samples from each participant and to simulate conditions expected to be experienced by the samples.

Samples for blood may be collected in a number of manners as is known to one of ordinary skill in the art. Numerous devices exist to collect blood samples, which may be stored in various manners, such as dry samples on filter paper, within an aliquot tube, within a capillary tube, etc., as a nonlimiting example.

### Capillary Blood

These samples may each be collected via a finger prick or arm-based device, wherein a lancet is utilized to pierce the skin and allow blood to flow into the collection device. Such finger prick styles are routinely used by patients around the world for obtaining blood samples. Each real-world finger prick blood sample had a matched reference venipuncture/peripheral blood sample collected by a phlebotomist, whether as a finger prick sample collected contemporaneously (such as within the same 60-minute period) to the other samples, or as a venous draw, also contemporaneously from the same donor on the same day for model accuracy determination.

FIG. 1 shows a method (100) for building the training data set for creation of the stability model. The kinetics of environmental parameters were determined for sample transport to the laboratory by post using a data logger fixed to a postage satchel. The minimum and maximum transit time was between less than one and eight days with a mean of 1.5 days. Replicates of donor samples were subjected to varying degrees of each environmental attribute in vitro, akin to those experienced by real-world samples (105). Sample aliquots were exposed to static and dynamic temperature fluctuations, repeated over multiday cycles, with and without agitation to simulate anticipated transit conditions. Dynamic temperatures, consisting of a sine wave temperature ramp between the minimum and maximum temperatures experienced over the day/night cycle at the peak of summer and the lows of winter, were included to simulate the effect of sample transport in an uncontrolled environment. Conversely, static temperatures were also utilized to replicate samples transported under common controlled conditions including, ice at a temperature of 4° C., cold packs at a temperature of 15° C., and ambient at a temperature of 22° C. Modifying the time, temperature and agitation attributes experimentally were intended to capture the permutations of environmental conditions (105) a blood sample could experience during transit.

Referring to FIG. 1, method (100) is used to build a training data set for creation of stability models. In this particular model formation, donors (101) provided a venous blood sample (102), each of which was divided (104) into a plurality of aliquots (for example, about fifty to one hundred aliquots per donor) for testing and simulation of environmental attributes. Donor and health attributes (103) such as age, sex, body mass index (BMI), medication(s) and/or medical condition(s), are a characteristic of the individual providing a sample and which were collected at the time of sample collection. Data on donor and health attributes can be collected before, during, and/or after the time of collection and sent alongside the venous blood samples (102).

A first subset (104a) of aliquots of each sample were subjected (106) to simulated postage conditions including static and/or dynamic temperature exposure and agitation. As used herein, an environmental attribute (105) is an external force applied to the sample while in transit, including but not limited to time, temperature, vibration, pressure, light, etc. Data on environmental attributes can be captured during transit by accompanying each sample with sensors and/or data loggers included with the postage packaging, or can be obtained by a proxy, as provided in some embodiments.

One or more data loggers were placed in close vicinity to the blood sample and accompanied the samples during transit to the test facility to monitor the environmental conditions experienced by the blood sample during transit. Here, “environmental conditions” means one of time and/or temperature that the blood sample is subjected to since collection. The one or more data loggers includes one or more of: (i) a temperature sensor for detection of temperature fluctuations of the sample during transit and (ii) an accelerometer for detection of impact/vibrations or kinetic shocks experienced by the package and the sample during transit since kinetic agitation of the sample can accelerate the lysis rate of the blood cells. Additional sensor equipment that may be used to accompany the test samples during transit include: a light sensitivity sensor to monitor any potential exposure of the sample to light, and/or a geolocation sensor to track the sample during transit for extrapolating environmental conditions from weather data such as local temperatures experienced by the package.

Degradation attribute (107) is an internal sample metric, such as evaporation, cell lysis, plasma hemoglobin, plasma DNA content, or a proxy for degradation as described herein, which indicates to what extent a sample has degraded during transit. Data on degradation attributes (107) are enumerated from the sample upon receipt in the laboratory. Alternatively, models that predict degradation from environmental conditions can be used in place of direct quantification of degradation attributes from the sample.

Environmental attributes (105) are known to affect how blood changes during transit. Here, it was investigated whether environmental attributes (105) (for example, agitation, time, temperature, light exposure, and/or evaporation), degradation attributes (107) (for example, cell lysis, plasma hemoglobin, plasma DNA content, and/or degradation prediction) and donor and health attributes (103) (for example, sex, age, ethnicity, and BMI, medical condition[s], and/or medication[s]) were significant covariates of analyte change to establish the stability model's functions.

A measurement (108) of a plurality of analyte characteristics is then made for the degraded samples and the samples are then subjected to plasma extraction (110). A second subset (104b) was not subjected to the degradation from environmental attributes (105) or degradation attributes (107) but rather passed directly to the plasma extraction step (110). Analyte measurements of the direct samples (104b) are considered to be the true value (109) of the analyte characteristic for each analyte measured. Analyte measurements of the degraded sample subset (104a) are considered to be the test value (111) of the analyte characteristic for each analyte measured. The test values (111) for each analyte measured are compared (112) to the analyte measurement from the true values (109) to determine the relative difference between the true and test values to construct a regression line capable of predicting a correction factor in order to create a training set (140) to be able to train (114) a stability model (150) to predict the relative change of the analyte measurement with respect to the donor and health (103), environmental (105), and degradation (107) attributes.

Similarly, in FIG. 2 a process was performed using capillary blood collected by finger prick blood samples to validate the model. As described in FIG. 2, matched venous and finger puncture samples were collected from donors. The finger puncture samples were sent to the laboratory through the post for analysis. The venous puncture sample was immediately processed and stored. However, Applicant notes that a similar result can be achieved without the validation data of FIG. 2, or that the training set could also be solely derived from the process of FIG. 2. Therefore, any combination of training and validation sets can be used to train the models, as a greater number of data typically increases the accuracy of the model.

Here, donor (201) provides their donor and health attributes (203), and a venous sample (204) is collected for a true value, and a finger prick sample is contemporaneously collected (202). The finger prick sample is then subjected to mail service. Method (200) is designed for validation of the applicability of the correction model and to assess the model performance against real-world samples transmitted via the postal service. Finger prick blood samples were collected (202) from thirty-seven donors (nineteen male, eighteen female with a median age of forty-five and an age range of twenty-one to seventy-one) (201) and sent (206) through the post to the testing laboratory. Each posted sample was packaged along with a mobile/remote sensor and data logger (250) to monitor and record the environmental attributes (205) experienced by the posted sample during transit. Donor and health attributes (203) for each donor were collected either at the time of sample collection or by alternative means, for example, by a medical professional or the referring doctor, a phone interview with the donor or a digital survey completed by the donor. Each real-world finger prick blood sample had a matched reference peripheral (venous) blood sample collected (204) by a phlebotomist from the same donor (201) on the same day for model accuracy determination. In some cases, multiple real-world finger prick blood samples were provided.

Once received by the testing laboratory, the data from the sensor and sample transit period is downloaded (208), the posted blood sample degradation indicators measured (210) the in-transit environmental attributes (205) detected from sensor and logger (250) and associated with the posted blood sample and measurement (210). Degraded posted samples are subjected to plasma extraction (212) to determine the test values (211) of analyte characteristics for the blood samples. Simultaneously collected venous blood samples (204) were subjected directly to plasma extraction (212) and analyte measurement to determine the true values (209) for the venous blood samples (204).

Using the trained stability model (150) for each analyte of interest, a prediction (214) of the correction coefficients to adjust the test value (211) to obtain a corrected value (213) for the measured analyte. Corrected values (213) for each measured analyte from the degraded blood samples are then compared with true values (209) obtained from the undegraded venous blood samples (216). The % accuracy was determined between the corrected value and the true value (venous puncture) which used the formula of % accuracy=(corrected value−true value/true value)×100. The % accuracy for uncorrected was determined between the test value and the true value (venous puncture), which used the formula of % accuracy=(test value−true value/true value)×100. Such values can help to identify the rates of error of the models.

### a Quantitative Correction of Test Results

A critical improvement in the present disclosure that overcomes the significant limitations in the prior art is the use of a model of sample stability to make a quantitative correction to a test value (i.e., 5.5 mmol to 6.75 mmol) instead of just defining qualitative limits upon sample analysis (i.e., pass/fail). The present disclosure collected thousands of samples and subjected them to different environmental factors, to create the data training set created by the method of FIG. 1.

### Environmental Attributes

In view of the degradation of analytes occurring, when considering a degraded sample, a first critical piece of information is the time between when the sample is collected and when the sample is analyzed. As previously noted, some analytes within a given blood sample will have a greater level of stability than others. And, at the heart of the issue, the goal is to not simply to qualify whether a sample can be analyzed within some level of accuracy, but to actually quantify a given desired analyte, and have that quantified result be as close to the true value of the analyte, as if the analyte was analyzed immediately after collection.

Because a total of eighty-seven analytes were tested via the models (Table 2), determining what variables to collect and how to weigh such variables becomes essential. There are several methods for selecting variables in machine learning: (i) filter methods select features based on statistical properties, such as correlation with the target variable, variance, information gain, mutual information, and chi-squared tests; (ii) wrapper methods use a specific machine learning algorithm to evaluate the performance of different feature subsets; and (iii) embedded methods combine feature selection with the model building process, or a combination of many methods. Variable weighting or fitting coefficients refers to the process of finding the optimal values for the parameters of a model in order to minimize the error or loss function, with methods including: (i) gradient descent and optimization algorithms used to minimize the loss function by iteratively updating the model parameters in the direction of the steepest descent of the gradient; (ii) closed-form solution allow for directly calculating the optimal values of the coefficients; (iii) regularization used to add a penalty term to the loss function, which helps in preventing overfitting and controlling the complexity of the model; (iv) Bayesian inference estimates the posterior distribution of the model parameters given the data; and (v) cross-validation can be used to evaluate the performance of the model and finetune the coefficients by selecting the best hyperparameters through repeated training and testing on different subsets of the data. The choice of these methods depends on factors such as the size of the dataset, the nature of the data, the computational resources available, and the specific goals of the machine learning project and should be determined by someone skilled in the art.

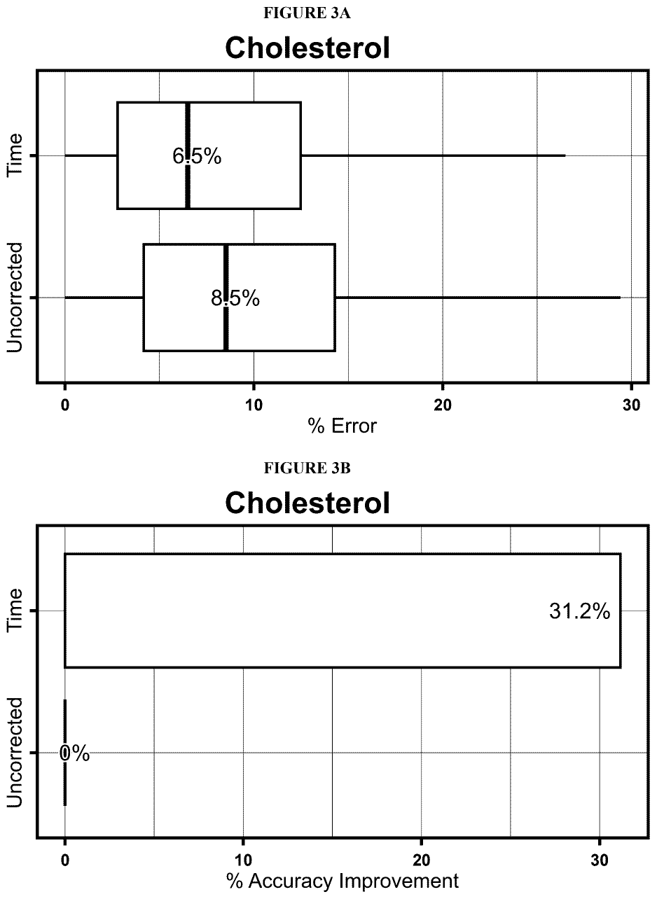

The environmental factor, time, was deemed to be a singular variable that was important for many of the analytes tested. Of the eighty-seven analytes that Applicant tested, 76% showed an improvement in test accuracy when using just the time variable. FIG. 3A depicts a bar plot showing the error associated with an uncorrected test result as the control, (i.e., no time consideration for the samples), and a test result corrected using a time variable for cholesterol. The bar plots within the figures define a median point, which is shown as a vertical line within a box, with the given value being the median point. The box defines a 25% to 75% range, with the lines extending from the same as the outliers of the minimum and maximum values. Notably, by making a correction according to time, the sample shows a significant improvement in the error rate. An example of a linear regression equation used in FIG. 3A to correct the change in test results would be, Δtest=β×sampleTransitInDays. FIG. 3B depicts a bar plot showing the improvement associated with an uncorrected test result as the control and a test result corrected using a time variable for cholesterol, which yields a 31.2% improvement in the error rate. Such a correction is incredibly important for virtually any sample.

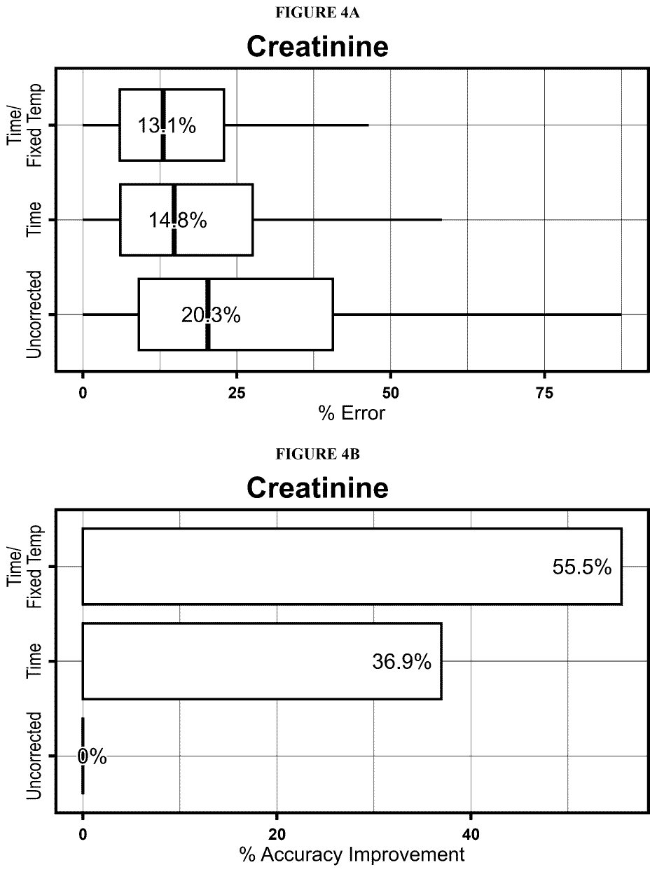

Beyond the inclusion of time within the stability model, FIGS. 4A and 4B detail the improvement of a quantified blood test of an analyte using time or time and a fixed temperature control. Fixed temperature is a common storage strategy. For example, if a blood sample is taken and then mailed in an insulated box, with or without a heat or cold pack, the temperature would likely be stable at that given temperature, at least as compared to an uninsulated box. Applicant tested such examples, including a temperature at multiple points using such stable temperatures. Applicant also provided certain samples that varied the temperature of the sample over a given time period, but averaged the temperature over that time period, providing an equivalent to a fixed temperature assumption. Such an average temperature might be obtained by taking a temperature from a data logger at every one-hour interval, and then taking the sum of the total temperature and dividing by the total hours to obtain an average. A very simple example would be over five hours of time, a temperature of 10° C., 10° C., 15° C., 20° C., 20° C., would provide for an average of 15° C., and would provide the same total temperature as if the sample was at a static 15° C. over five hours.

Inputting the data into our model, of the eighty-seven analytes that Applicant tested, 85% showed an improvement in test accuracy when using the time and fixed (average) temperature variables. An example of a linear regression equation used in FIGS. 4A and 4B to correct the change in test results would be Atest=BxsampleTransitInDays+α×meanTransitTemperature. Taking creatinine as the example analyte, a further 1.7% improvement in absolute error is shown when adding fixed time to temperature in FIG. 4A and as depicted in FIG. 4B, the average improvement of test accuracy relative to the uncorrected was approximately 55.5%, which is 18.6% better than using time alone.

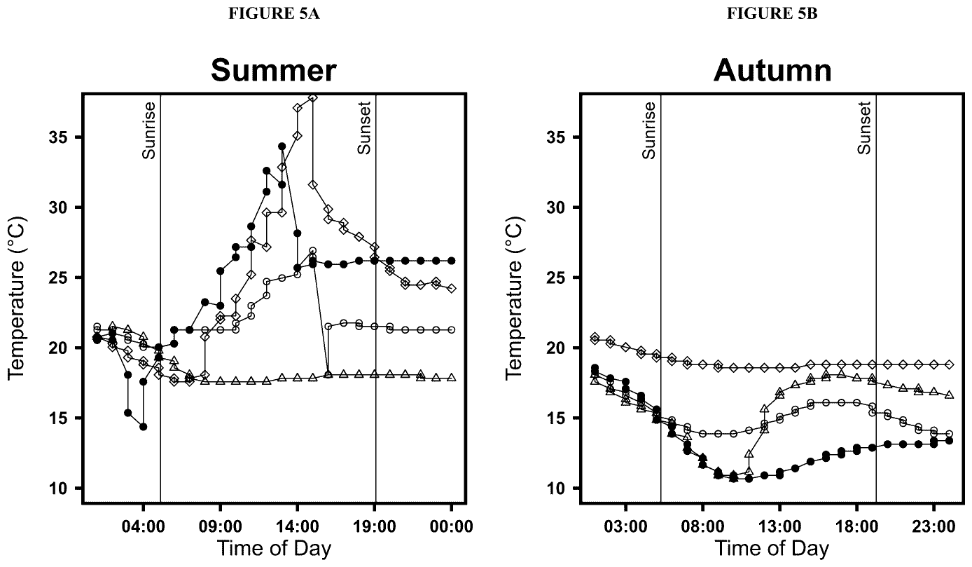

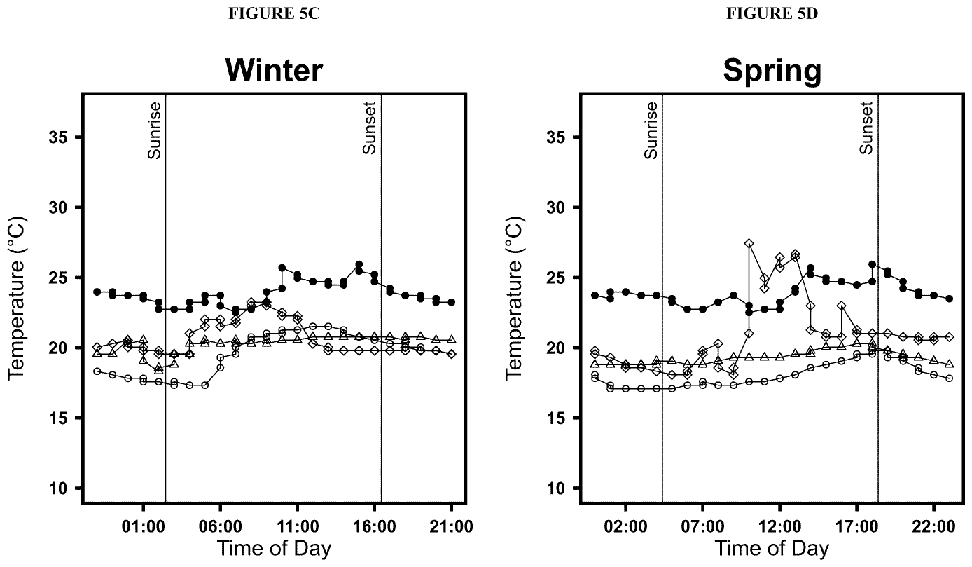

However, temperature in the real world is virtually never static, particularly in an uncontrolled environment such as the mail. FIGS. 5A, 5B, 5C, and 5D depict seasonal temperature plots and their variance for real-world samples transport by mail, with temperature captured by a data logger accompanying each sample. The different plot lines in each of FIGS. 5A-5D are simply showing different samples. Therefore, a fixed temperature is not an accurate representation of the conditions of sample transport in an uncontrolled environment, in which sudden changes in conditions affect test accuracy. Applicant monitored the temperature during transit for over six hundred patient samples in Australia over a two-year period between Aug. 16, 2021, and Nov. 3, 2023. Temperature values recorded during transit ranged from below freezing to 43.5° C. (not all data shown), varying with seasonal and diurnal cycles. The temperature of a blood sample during transport by mail is dynamic, i.e., not a controlled temperature, and no temperature control has been attempted. Therefore, the samples will effectively be at the ambient temperature through the mailing journey.

Of course, it is known to those of ordinary skill in the art, that temperature can and will impact the rate of shift in degradation, and thus impact the quantification of an analyte. Thus, a sample held at 4° C. at a static temperature for twenty-four hours would almost certainly undergo a different shift and degradation than a sample held at −10° C. for twelve hours and then held at a temperature of 18° C. for another twelve hours, even though the total temperature would be equivalent over the time period. Indeed, in such an extreme example, the freeze/thaw temperature change would likely further impact the sample, even though the total temperature experienced by the two samples would otherwise be mathematically equivalent if Applicant simply averaged the temperatures.

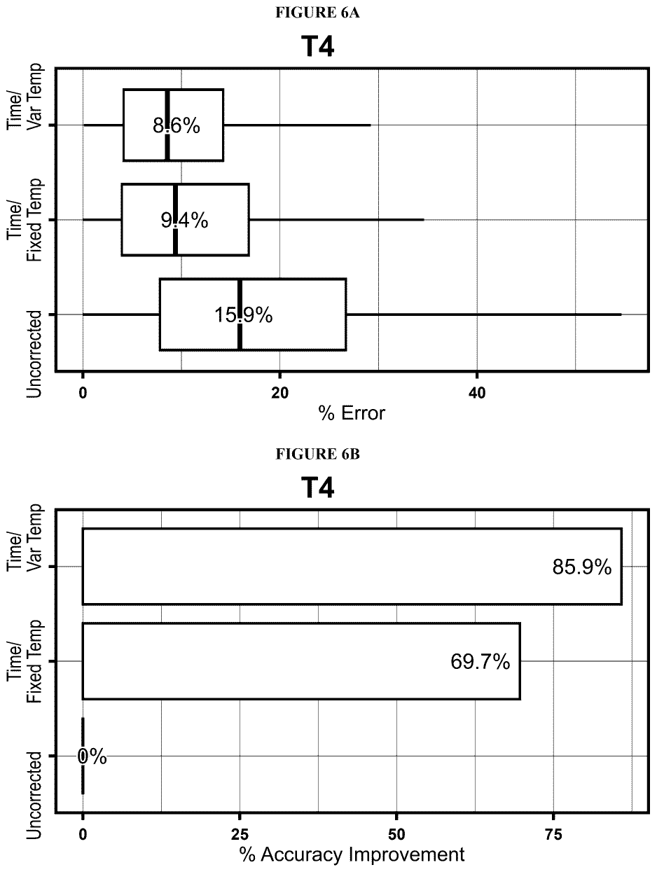

Of the eighty-seven analytes that Applicant tested, 88% showed an improvement in test accuracy when using the time and mean, minimum, and maximum (variable) temperature variables. In both FIGS. 6A and 6B, variable temperature was recorded using a data logger. FIG. 6A shows a model of sample stability that accommodates a variable temperature and improves test accuracy for samples transported in an uncontrolled environment. This figure depicts a bar plot showing the error associated with uncorrected test results as the control, test results corrected using time and a fixed temperature, and test results corrected using time and a variable temperature for thyroxine (T4). In FIG. 6A, there is an absolute change in error of 0.8% between the time and fixed temperature and the time and variable temperature, but this provides for a total improvement of nearly 16.2% between the two correction methods as detailed by FIG. 6B. An example of the linear regression equation used in FIGS. 6A and 6B to correct the change in test results would be, Δtest=β×sample TransitInDays+α×meanTransitTemperature+δ×min TransitTemperature+γ×maxTransitTemperature. Minimum and maximum temperature features were included as examples of the accuracy improvements that can be engineered from a time series of environment data. The Applicants acknowledges that environmental data that possesses a temporal attribute could be used in its entirety or summarized in different ways to achieve a performance improvement in addition or in place of minimum and maximum temperature. Common ways to summarize temporal data include but are not limited to: (i) maximum drawdown that measures the maximum loss from a peak to a trough of a time series data before a new peak is attained; (ii) standard deviation of the time series data can provide information about the volatility or variability of the data points around the mean; (iii) percent change between consecutive data points can help identify the magnitude of changes over time and detect trends or anomalies; and (iv) relative strength index is a momentum oscillator that measures the speed and change in a time series data.

The variable temperature plots in FIGS. 5A-5D were obtained by including the aforementioned data logger, having a temperature probe during transit. However, adding a data logger to each sample may not be possible or reliable. First, this may introduce an extra layer of human error in using the data logger, starting it properly and/or locating it properly within a sample package to obtain a valid and viable temperature reading. Secondly, this greatly increases the cost of shipping the package and/or increases the total costs in processing the samples, simply due to the cost of data loggers and obtaining information from them when processing data. Finally, a broken data logger, or a malfunction that provides for a false reading would add significant error risk into a sample calculation. Applicant therefore devised a strategy to approximate the temperature of the sample that would not require the inclusion of a data logger.

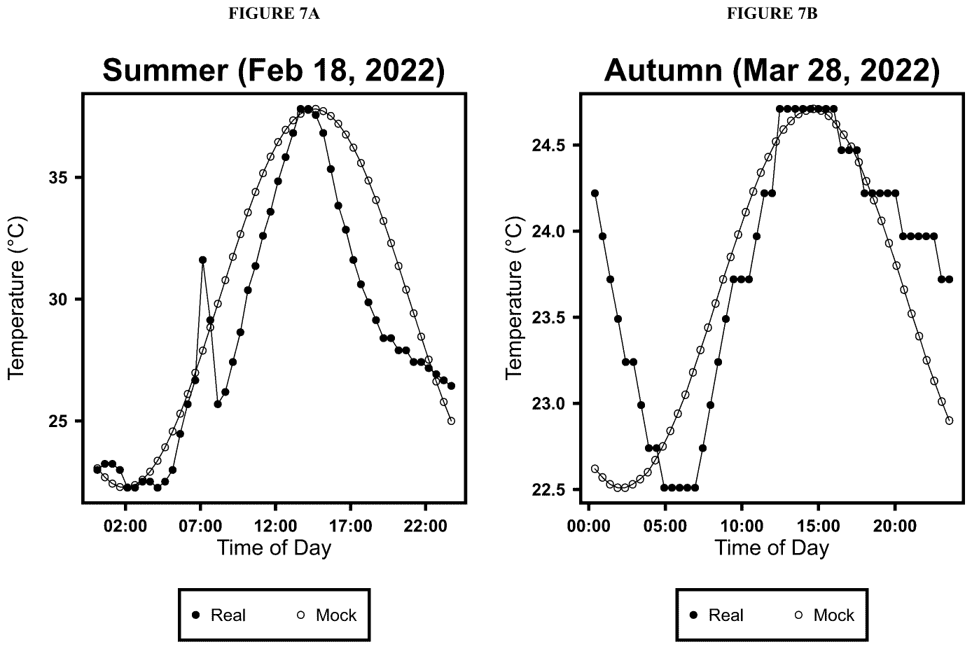

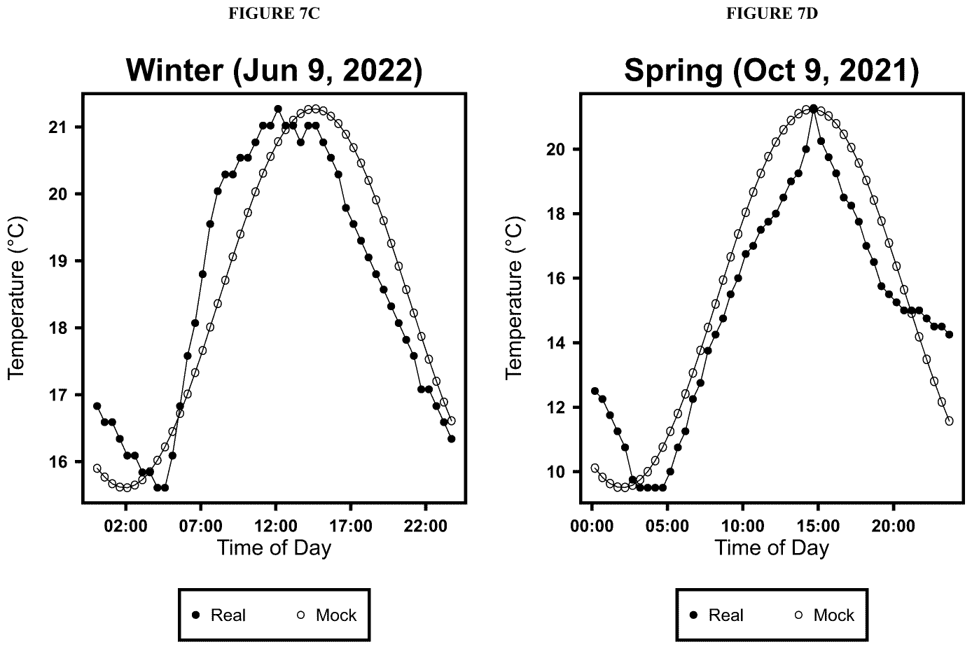

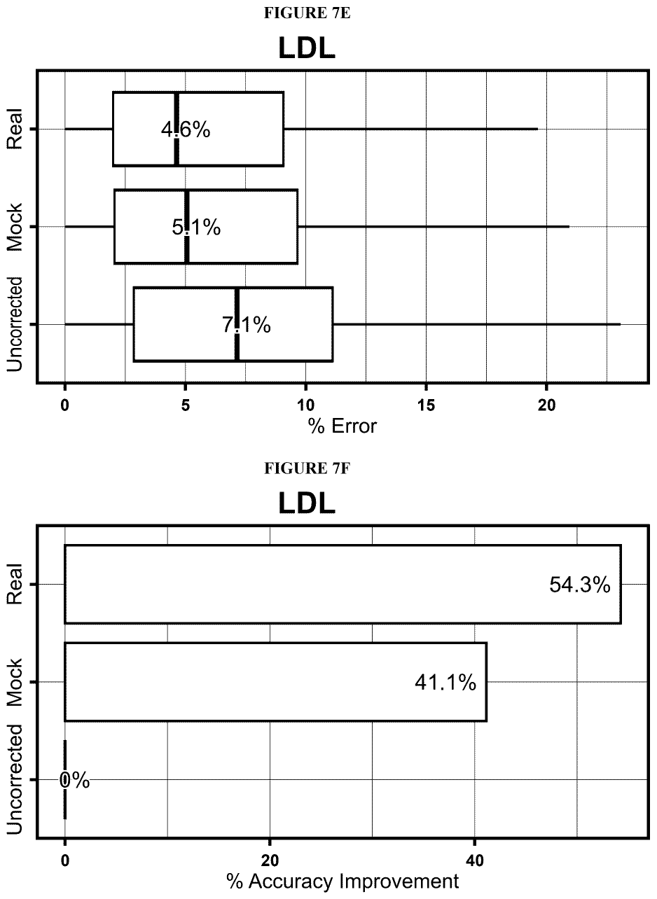

Applicant found that using publicly available weather data to approximate the real-time temperature tracking of data loggers yields a highly correlated improvement in test accuracy, virtually identical towards using a data logger per se. Using publicly available weather data on minimum and maximum temperatures over the period of transit, it is possible to approximate data from the temperature sensor that accompanies each blood sample. The collection of this publicly available weather data can be collected by many mechanisms, from manual collection and searches to performing an API call by the system or any other generally know ways of collecting data to one skilled in the art, requesting the temperatures from the area of transit and the area of receipt, and providing the input for average, min, and max temperature as outlined herein. This data can then be used to approximate the temperature changes over the transit time. A plot of a real collection of temperature as compared to an estimated temperature (mock) is provided for each of the four seasons in FIGS. 7A, 7B, 7C, and 7D. Surprisingly, the mock data provides a highly correlated estimate of the actual temperatures across the various temperatures in each season. Indeed, the mock data was a highly unexpected finding, as there was an expectation that the samples might face a greater randomization in transit, such as within hot vehicles or buildings, or conversely in a temperature limited building instead of ambient temperatures. Instead, the correlation was so strong that mock temperature provides a suitable replacement option for a data logger.

Because of this strong correlation shone in FIGS. 7A-7D, a model of sample stability that accommodates a mock variable temperature to improve test accuracy for samples transported in an uncontrolled environment was thus devised and tested. FIG. 7E depicts a bar plot showing the error associated with uncorrected test results as the control, test results corrected using a variable temperature as determined by a temperature data logger (FIGS. 6A and 6B) designated as “real test results” corrected using a variable temperature as determined from publicly available weather information designated as “mock.” Of the eighty-seven analytes tested, 83% showed an improvement of test accuracy when using the mock variable temperature variables. FIG. 7F depicts a bar plot showing the % accuracy improvement associated with uncorrected test results as the control, test results corrected using a variable temperature as determined by a temperature data logger and test results corrected using a variable temperature as determined from publicly available weather information for LDL. The average improvement of test accuracy when using mock variable temperature relative to the uncorrected was approximately 41.1%, whereas using the actual measured temperatures yielded a total improvement of 54.3%. This small difference still provides for a meaningful improvement over the alternative and provides for a good proxy solution.

This predicted temperature strategy (mock) provides for an improved user experience, as it prevents error by inclusion of an additional device with a donor sample. Furthermore, users may also benefit by cost reductions, and it also eliminates errors where the data logger malfunctioned, was misplaced, or was otherwise improper due to user error. Therefore, as compared to assumption of a static temperature, an improved model uses predicted temperature data from accessible databases.

### Donor Attributes

Further incremental improvements to the model can be made by adding additional data points. For example, donor information is also a critical parameter for many analytes. When considering the blood sample, one key feature, of course, is the donor themselves. Applicant found a strong correlation between the sex of the donor, and/or the donor's age and model accuracy. When considering a blood sample, one of the biggest differences in certain components of the blood is the presence and/or absence of certain hormones. These may be produced by the sexual organs and in other glands, such as the adrenal glands. Sex hormones are transported in active and inactive forms, bound to carrier proteins that moderate activity, but also the rate of their degradation and consumption. The quantity of such hormones and related analytes can implicate the rate and drift of additional analytes within a sample. Thus, the sex of the donor can be utilized to estimate such changes, and its inclusion into the model improves outcome. Genetic differences existing between sex and age can affect metabolism and the rate of sample degradation. Furthermore, where available, the donor's ethnicity can also be considered, which may impact the rate of sample degradation.

The donor's age is likely to have a significant effect on sample degradation based on the presence, absence, and/or concentration of certain hormones and the changing ratio to inhibitory carrier protein that moderates the rate of degradation and consumption. A male aged twenty-five will have dramatically different hormone levels than a male of age seventy-five. Similarly, women of childbearing ages, again, such as a twenty-five-year-old woman, would have vastly different circulating hormone levels than a woman aged seventy-five. Thus, age logically can impact the presence and absence of certain hormones, their concentrations, and thus the likely drift of a given analyte within a sample.

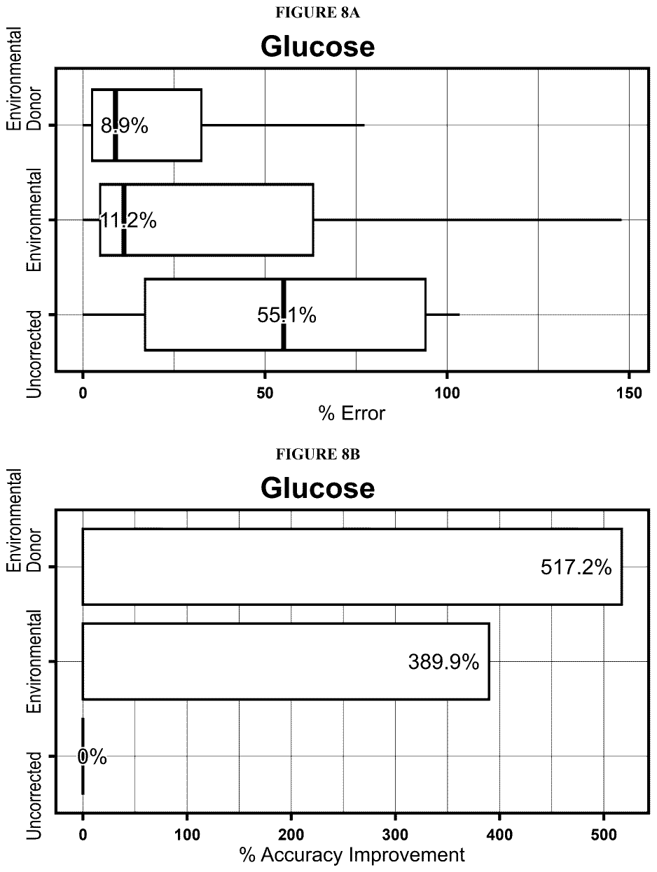

FIGS. 8A and 8B depict the improvements in a quantitative correction to a test value and the inventive step made by inclusion of donor attributes. Of the eighty-seven analytes that Applicant tested, 92% showed an improvement of test accuracy when adding in donor parameters. FIG. 8A depicts a bar plot showing the error associated with uncorrected test results as the control, test results corrected using only environmental attributes (e.g., time and/or temperature) and test results corrected using environmental attributes plus donor attributes (e.g., sex and/or age) for glucose. Thus, FIG. 8A depicts that use of a model of sample stability that accommodates patient demographic information (e.g., sex and/or age) improves test accuracy for samples transported in uncontrolled environments. An example of the linear regression equation used in FIG. 8A to correct the change in test results would be, Δtest=β×environmentalAttributes+α×donorSex+γ×donorAge+δ, and optionally including×donorEthnicity, where available.

FIG. 8B depicts a bar plot showing the % accuracy improvement associated with uncorrected test results as the control, test results corrected using only environmental attributes as previously noted and test results corrected using environmental attributes plus donor attributes (sex and age) for glucose. Here, in FIG. 8B the power of the additional donor attributes can really be seen, as the improvement with the addition of these donor attributes changes the accuracy improvement percentages by more than 500% compared to the uncorrected test results and 127.3% compared to the test results corrected by only environmental attributes. Indeed, the inclusion of such data from the donor, and its use in models to quantify analytes is totally unexpected that such a dramatic change would be present by these minimal inclusions alone.

The combination of a model that includes at least one environmental factor, such as one or both of time and temperature, then in combination with donor information, provides for dramatic improvements for the stability model. These factors are also important as they are the factors that are already required to be collected for virtually all samples, as it will be apparent of the time, and therefore also the temperature (when obtained via a proxy or from a data logger) for the sample, and in order to provide the information to the proper patient, the donor's age and sex will also be available in virtually all cases.

### Sample and Degradation Attributes

Sample and degradation information is also a critical parameter for many analytes. There are intrinsic features within a sample that act as an internal clock and correlate with the degree of sample degradation relative to the accumulation of extrinsic forces acting upon the sample. Sample-specific attributes, including but not limited to sample volume and plasma: hematocrit ratio also influence the rate and magnitude a sample degrades. When blood samples are collected by a patient outside of a laboratory or medical setting, typically, those samples are not immediately separated into plasma and red blood cells. This is a direct difference in how such samples are processed when a sample is taken at a laboratory or hospital setting. Consequently, issues such as the lysis of blood cells can occur due to a delay in sample processing of the whole blood samples, and if present, can cause erroneous peaks in analytes concentration or interfere with the measurement of certain analytes. Applicant hypothesized that the detection of factors that are typically absent or in low abundance in an undegraded sample associated with sample degradation can be utilized to further improve the model and to predict samples which may be degraded beyond the expected.

Hemoglobin (Hb) is carried by RBC in blood and is not typically found in plasma that is collected and stored correctly. Lysis of RBC as a consequence of incorrect collection and storage of a blood sample can cause Hb to be released into plasma upon cell lysis, resulting in a visual color change. Hb can be measured directly with specific reagents or indirectly through the color change by spectrophotometry using the Harboe method and any of the many derivative methods. Detection of Hb in plasma above background level coincides with timing and magnitude of the color changes in the plasma and variation in environmental attributes including time, temperature, and impact/agitation (not all data shown).

In addition to Hb, other degradation attributes were found to provide incremental improvement in model performance. White blood cells (WBC) have differing osmotic tolerances, lysis rates and cytoplasmic contents to RBC that will inform the rate and sensitivity of sample degradation in a manner different to Hb. WBCs also do not contain Hb and therefore Hb cannot be used as an indicator for lysis of WBCs. As RBCs do not contain DNA, it was further hypothesized that DNA present in plasma could be a proxy for lysis of WBC that is complementary to Hb for lysis of RBC. Indeed, detection of DNA in plasma above background level coincides with timing and magnitude of the WBC lysis in the plasma and variation in environmental attributes including time, temperature, and impact/agitation.

WBC counts performed using trypan blue and a hemocytometer confirmed the lysis of WBC in a time- and temperature-dependent manner for peripheral blood mononuclear cell (PBMC) and polymorphonuclear leukocytes (PMN) counts after exposure to different temperatures for extended periods of time (data not shown). DNA levels in plasma had a discordant pattern to Hb plasma levels to support the notion that the inclusion of multiple degradation attributes can provide complimentary and additive information to improve model performance. Plasma DNA content can be measured directly with DNA-chelating dyes and reagents or indirectly spectrophotometry using the 260:280 ratios of nucleic acid or any of the many derivative methods.

Signals of sample degradation can be measured directly and indirectly, as described for the Hb and DNA examples above, but can also be predictions from other models. Instead of quantifying Hb by spectrophotometry, Applicant is able to train a model that predicts changes in Hb. This predicted Hb of an arbitrary unit, approximates the absolute Hb value quantified by spectrophotometry, without ever having to measure Hb. Consequently, a prediction of Hb can be used in place of real Hb observations as an input to train a model. Applicant can go further, knowing that cells when they lyse release electrolytes such as sodium, chloride, and potassium. The present invention is able to train separate models to predict changes in sodium, chloride, and potassium and append this new data to the existing data to increase the complexity. As models for sodium, chloride, and potassium are biologically linked and exhibit strong correlation with sample degradation, including output of these models improves predictive power and subsequently test accuracy. This approach is often referred to as model stacking, where the outputs from primary (base) models are used as input to train a secondary (stacked) model. Model stacking improves performance of weak models in these circumstances in two ways: (i) it incorporates predictions from stronger models that correlate better with sample degradation; and (ii) it adds new data variables that account for previously unexplained differences.

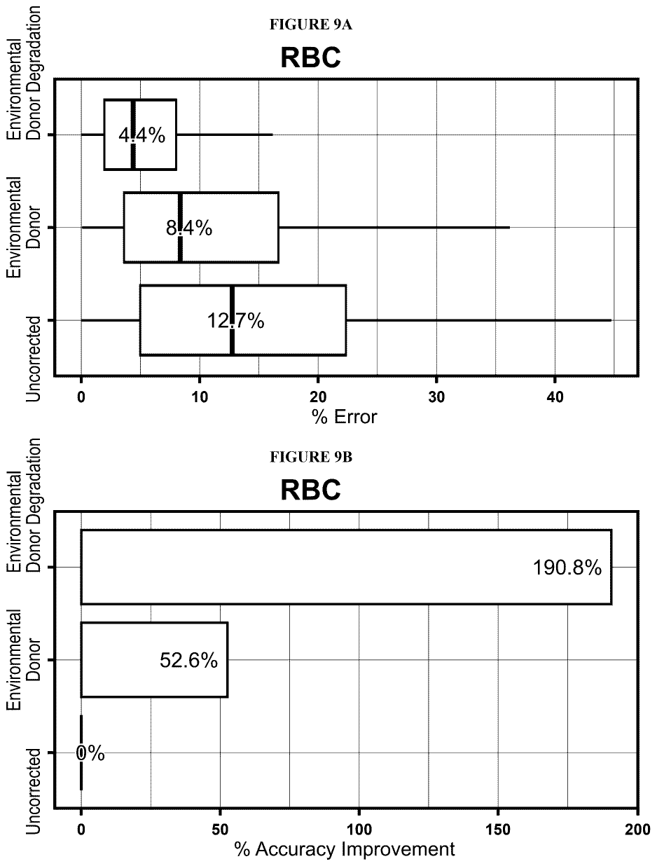

In FIG. 9A shows the accuracy improvement by using predictions of sample degradation attributes and stacking models of predicted degradation to train a model. In this example, some of the outputs from a first model can be used as predictors of the degradation, and thus provide for inputs as a proxy for degradation, when training a secondary model. Of the eighty-seven analytes that Applicant tested, 98.6% showed an improvement in test accuracy when considering degradation proxies by stacking models to train a secondary model. In FIG. 9A there is an absolute change in error of 8.3% between the uncorrected and environmental donor with degradation and an absolute change in error of 4.4% between the environmental donor with degradation than without. The average improvement of test accuracy relative to the base model was approximately 190.8% (FIG. 9B).

Therefore, Applicant noted significant performance improvements in a quantitative correction to a test value and the inventive step made by inclusion of sample and degradation attributes. An example of the linear regression equation used to correct the change in test results would be, Δtest=β×environmental Attributes+α×donorAttributes+γ×hemolysis+δ×predicted plasma chloride.

### Health Attributes

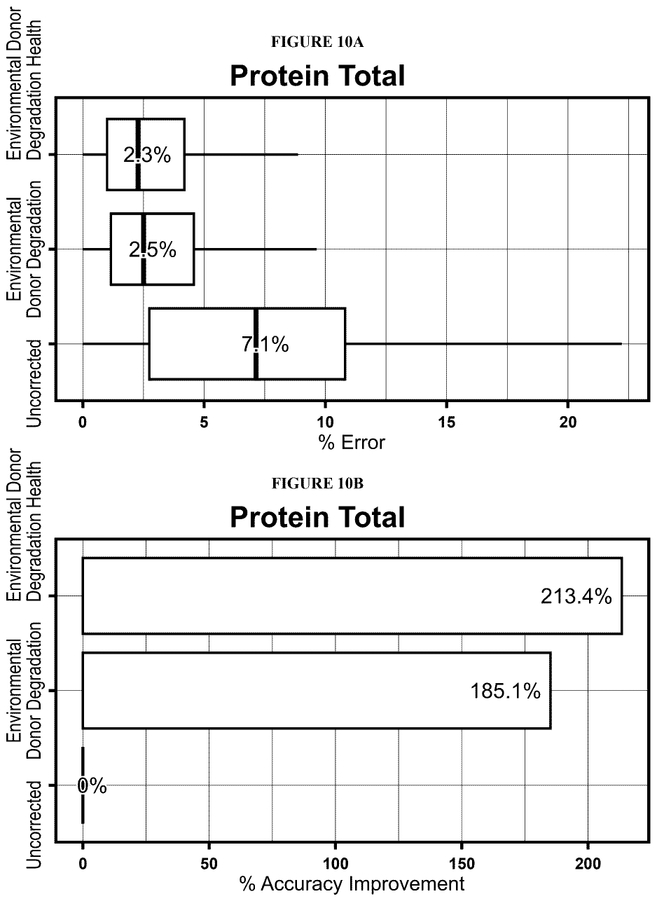

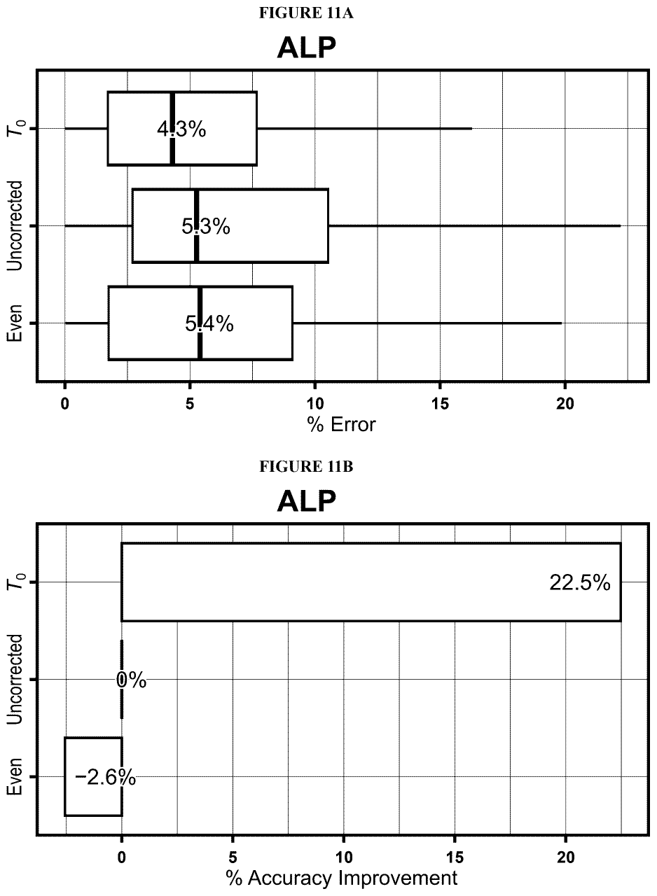

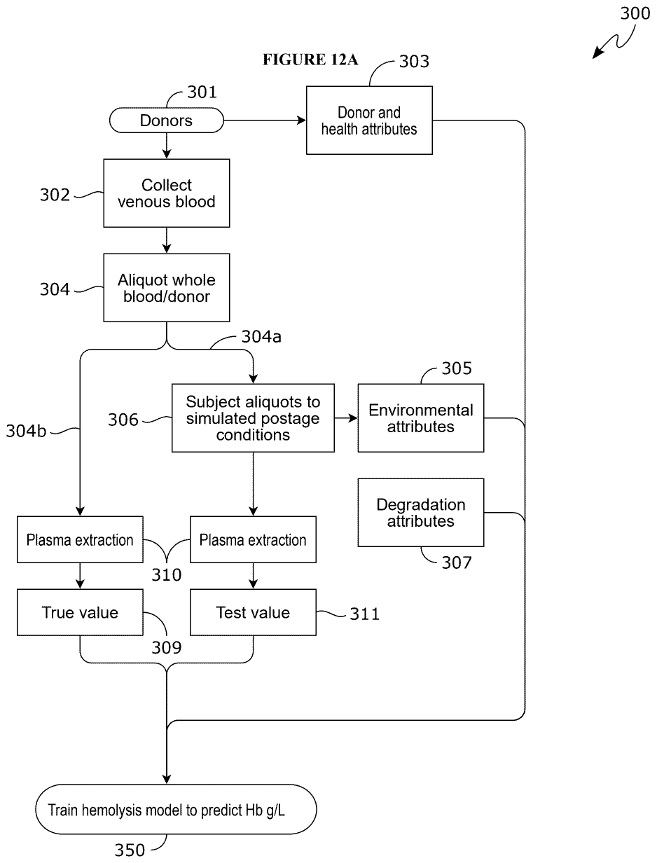

Additional features that may be optionally included within the model are additional donor features that reflect a patient's health. Again, as detailed above, the inclusion of certain donor characteristics can greatly increase the accuracy of the model when calculating an analyte from a degraded sample. FIGS. 10A and 10B detail that the inclusion of further donor information may provide additional incremental improvements to the model for certain analytes. For example, in certain cases, a blood sample may be requested from a patient, and so the patient data will be available for inclusion into the model. Such data can be, for example, exported from an electronic medical record (EMR) or otherwise collected by the caregiver requesting the blood sample. In this manner, specific information regarding the donor will be known.

The Applicant hypothesized that the health and disease state of a patient would influence the rate of sample degradation and the resulting test accuracy. One could imagine that patients with diabetes, obesity or prescribed a drug that moderates lipid metabolism may have a different rate of degradation of lipid than that of a predominantly healthy and unmedicated individual. This is a particularly important consideration as the majority of blood testing is performed on patients with underlying medical conditions and any discrepancies in how stability models perform in health versus diseased patients, could create a disparity in test result accuracy if not controlled for patient health. Here, the additional health attributes labeled as “environmental donor degradation health” in FIGS. 10A and 10B may include the BMI of the donor, medication(s) of the donor, and medical condition(s) of the donor. While these may not be required of all analytes, for certain analytes such inclusion may dramatically increase the accuracy when such additional donor health attribute information is known. Thus, FIG. 10A depicts a bar plot showing the error associated with uncorrected test results as the control and by inclusion of additional donor health parameters including the BMI, medication, and medical conditions. Of the 18 analytes that Applicant tested, 78% showed an improvement of test accuracy when using the environmental, donor, degradation, and health variables. An example of the linear regression equation used in FIGS. 10A and 10B to correct the change in test results would be, Δtest=β×environmental Attributes+γ×donorAttributes+α×degradationAttribute+δ×donorBMI+θ×donorMedicationε×donorMedicalCondition. Donor attributes are an abbreviation of the donorSex, donorAge and donorEthnicity variables.

While the reduction of % error between environmental donor degradation without health attributes and with health attributes is only 0.2%, the use of these additional health factors as depicted in FIG. 10B, shows the average increase in test accuracy for analytes improved of 213.4% in accuracy by the addition of degradation attributes relative to the uncorrected environment donor degradation by using the stability model that accommodates patient health information (e.g., body mass index [BMI], medication, and medical conditions), as compared to an improvement of 185.1% when ignoring the donor health attributes.

The methods herein contemplate the addition of donor health attributes such as medications, medical conditions, and donor BMI would be sourced for the given sample from a providing doctor. Thus, a doctor determines a blood sample is desired and the necessary information can be obtained by access to the EMR or as added by the doctor when ordering the blood sample. Therefore, the sample itself would be provided with such information upon receipt and processing of the sample via the training set and model.

Putting together all of the steps, a practitioner orders a blood test. A donor sample container is provided to the donor, wherein the donor sample container comprises a unique ID. The unique ID is stored within a database, and the donor information is associated thereto. Upon receipt of the donor sample at a processing laboratory, the environmental attributes are determined from a data logger and/or other proxy and added to the database associated with the unique ID. The donor sample is processed, degradation attributes are determined from the sample itself, and run through a first model. The degradation proxy attributes are then provided with a subsequent model along with the environmental and donor information to yield a correction value. Finally, the test value of the desired analytes is calculated, and the correction value is applied to yield the desired corrected value. The corrected values can be uploaded or associated with the unique ID, thus providing the analyte values that were ordered from the blood test. In some of the embodiments, the environmental attributes are determined from a proxy, such as via a predicted temperature, a predicted time, or both. In some cases, the time is predicted based upon degradation factors within the sample itself.

### Introducing Model Performance Bias (Case Weight)

At the heart of all machine learning and artificial intelligence is a cost/loss function. When training a model to predict an outcome, the loss function in most cases is trying to minimize prediction mistakes (errors). Unless told otherwise, the loss function treats all prediction errors as equal. In reality, however, prediction mistakes are not all equal and some errors have more significant consequences than others. For example, you return a positive test for whooping cough, a potentially deadly disease to infants, when you are in fact negative, which may mean you can no longer visit your newborn nephew. However, if you return a negative test when you are in fact positive for whooping cough, you are unaware of the risk you pose by visiting your newborn nephew. These errors are not equal, as the consequences as the consequences of the mistakes are disproportionate. Applicant can control for this disproportionate impact when Applicant train a model by weighting data unevenly to penalize certain prediction errors more heavily. Essentially, Applicant can introduce a bias in a model to tune a desired performance for a specific use case.

Applicant hypothesized that using prior knowledge of the sample transit conditions for a given geographical region or postal/courier network, such as distribution of sample transit duration, can be used to skew model accuracy to ensure maximum performance for the most common sample transit conditions. This introduction of bias does so at the cost of lower performance for samples that arrive outside of the typical sample duration, however, over the majority of samples, this will result in a performance improvement. Applicant acknowledges that this method is not limited to weighting by sample transit duration but can be extended to any prior knowledge that can create a performance improvement at a population level.

Weighting certain samples more heavily than others, and such weighing has significant impacts on the total error percentages and the accuracy defined by the model. Poisson and exponential distributions approximate sample transit duration, i.e., the probability of a sample being delivered within a timeframe. Using this prior knowledge of the sample transit duration, Applicant weighted data using a Poisson distribution prior to model training. The process of case weighting using a Poisson distribution typically involved calculating the expected frequency of occurrence of an event, i.e., time taken to deliver a sample to the laboratory, and then using the Poisson distribution to assign weights that reflect the relative rarity or commonness of that event or characteristic for each case. Using days in transit as an example, twenty patients send samples by mail to the laboratory, fifteen samples are delivered within one to two days being the most common, followed by three days (three samples), four days (one sample) and five days (one sample), where more than five days is exceedingly rare, with zero samples. Using this information, Applicant assigned a value of fifteen (one to two days), three (three days), one (four days), one (five days), zero (more than five days). Consequently, during model training, accuracy for samples received in one to two days is five times more important than three-day samples, and samples received in three days is three times more important than four-day and five-day samples. A separate model can be trained based on days based on a range of days, for example, with a one- to three-day model, a two- to four-day model, a three- to five-day model, a four- to six-day model, or any other combination of days to give greater weight to the given sample by the number of days the sample was in transit. Using case weight to bias a model of the specific case of sample transit by mail, of the eighty-seven analytes that Applicant tested, 89% showed an improvement in test accuracy when case weights were used to skew model performance to a distribution of sample transit durations. Thus, a different model could be applied to a different sample based upon the number of days in transit, as a nonlimiting example, where a sample received in 2 days, would use one model and one received in 4 days, would use a second model, weighted for those particular occurrences.

Therefore, FIG. 11A depicts the use of a model of sample stability that applies variable weights to different data points improves test accuracy for samples transported under specific use cases (e.g., sample transported in an uncontrolled environment within one day of collection). FIG. 11A depicts a bar plot showing the error over one day associated with uncorrected test results as the control, test results corrected using a model trained on evenly weighted data and test results corrected using a model trained on data heavily weighted for To accuracy for ALP. The difference in this weighing is then further depicted in FIG. 11B showing the % accuracy improvement over one day (typical sample transit duration) associated with uncorrected test results as the control, test results corrected using a model trained on evenly weighted data and test results corrected using a model trained on data heavily weighted for To accuracy for ALP. The average improvement of test accuracy relative to the uncorrected was approximately 22.5% (FIG. 11B).

By introducing bias into the training of a stability model, we can dramatically improve performance for its intended use, to correct test results of a sample collected at home, that on average will be received within one to two days, or as received at three, four, or more days as acceptable for a given analyte test, so as to increase accuracy of the model to the given sample.

### Quality Assessment

At-home sample collection requires patients to collect, store, package and transport their own capillary blood sample without direction or instruction by trained medical professionals. Despite the collection kits being supplied with detailed instructions for blood collection that result in a successful collection by the majority of patients, around 10% will collect and/or store their sample in a manner not recommended that may affect sample quality and degradation. Data loggers are affixed to the kit packaging and not the sample collection tube itself, meaning that deviations in sample storage during collection by the patient are unlikely to be captured by the data loggers. As an example, a patient may temporarily store their sample in the freezer, but the collection kit containing the data logger is on their kitchen bench at room temperature. As a result, the environmental conditions recorded by the data logger are at odds with the sample and could lead to inaccurate test results if these discrepancies are not identified. This is particularly important for the embodiments described herein as the correction is reliant on the data logger accurately capturing the conditions of the sample collection and transit.

Laboratories have strict quality control (QC) and quality assurance (QA) in place to reject samples for analysis that are suspected of being incorrectly collected or stored. These thresholds are similar to the qualitative threshold used to reject samples based on time since collection (Table 1), but instead use predefined sample degradation thresholds. This process is in part reliant on indicators of sample degradation such as hemolysis of red blood cells (RBC) that cause a color change in the plasma. This degradation can be measured by clinical analyzers or spectrophotometer and samples can be rejected after a qualitative assessment. However, this approach is not fit for remotely collected samples as a certain level of degradation is expected given the extended period of time between sample collection and analysis. Consequently, many if not most remotely collected samples will be rejected and unable to be analyzed using existing processes (Table 1). Applicant's method of sample degradation quality assessment uses an environmental attribute (such as time and/or temperature) to infer an expected level of degradation. If the observed sample degradation is significantly discordant from the expected sample degradation, only then is a sample rejected, and a recollection of the sample is desired. This approach is scalable, extends the measurement window for test analytes, ensures sample QC assessment is dependent on each unique set of transit conditions and captures common collection and handling errors by patients inexperienced with sample collection, but allows for the majority of samples to still be processed.

Applicant has shown that the rate and degree of sample degradation is predictable based on at least environmental attributes. In other words, using information from the data logger Applicant knows how much degradation to expect in a given sample, and if we contrast this expected level to the observed level of degradation measured directly from the sample, Applicant can identify samples with a problem.

Furthermore, the Applicant hypothesized that this new method of quality assessment would help identify common mistakes made by patients inexperienced in sample collection. These included: (i) freeze and thaw storage of the sample; (ii) scraping blood from the finger into a sample collection tube; (iii) incomplete/ineffective blood mixing with the anticoagulant; and (iv) shaking or applying significant agitation to the blood. Unexpectedly, each of these mistakes made by patients causes a different pattern in sample degradation, many of which are undetectable by traditional QC methods. As sample degradation may cause higher levels of analyte than the endogenous levels in the plasma and misdiagnosis, it is important that any QC method for assessment of at-home collected samples be sensitive to these common issues for the purpose of sample rejection and to correct patient actions.

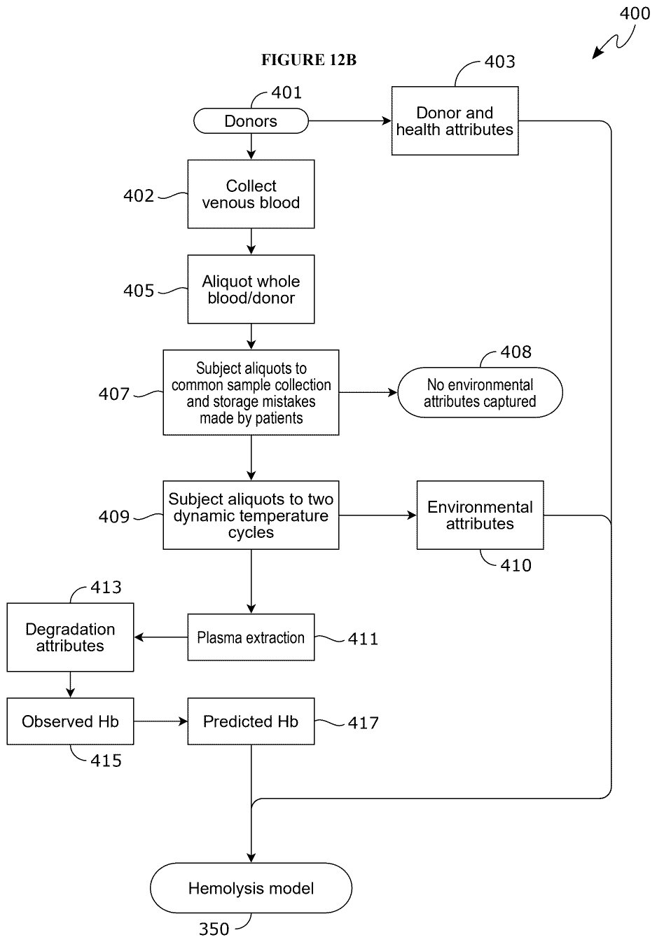

The hemolysis model was developed using method (300) of FIG. 12A in a similar manner to method (100) of FIG. 1 where venous blood (302) and donor and health attributes (303) are collected from donors (301) and aliquoted (304) where a first portion (304a) of the aliquoted samples is subjected to simulated postage conditions (306) combined with environmental attributes (305) prior to plasma extraction (310) to obtain test values (311) and a second portion (304b) is sent for immediate plasma extraction (310) to obtain true values (309) which are then combined with the degradation attributes (307) to train the hemolysis model to predict Hb g/L (350) as a measure of sample degradation.

The model development data set consisted of peripheral/venipuncture blood samples collected by a phlebotomist and subjected to temperature time courses. Aliquots of whole blood from each donor were stored as a reference sample or subjected to static or dynamic temperatures for a defined period of time. Static incubations were fixed temperatures, whereas dynamic incubations used temperature ramps to approximate the ambient changeover the day/night in the summer, spring, autumn and winter months. After each incubation period, plasma was separated from the blood, stored at −80° C., and thawed for quantification of analyte concentrations by multiplex immunoassay.

Again, similar to method (200) of FIG. 2, a blood sample degradation model validation is set by subjecting blood sample to common mistakes patient make when collecting and/or storing a sample. These conditions are included to simulate a discrepancy between the observed sample degradation and the expected degradation informed by the data logger. Similar to FIG. 12B where venous blood (402) and donor attributes (403) are collected from donors (401) and aliquoted (405) prior to being subjected (407) to common sample collection and storage mistakes made by patients without the benefit of known environmental attributes being collected (408). The validation data set consisted of a single donor, subject to four common sample collection and storage mistakes in quadruplicate. Aliquots were then subjected (409) to a plurality (for example, at least 2) of dynamic temperature cycles and combined with environmental attributes prior to plasma extraction (411) to determine the degradation attributes (413) associated with the known factors experienced by the samples in previous steps (407 and 409). The degradation attributes (413) were then associated with observed Hb levels (415) in the samples and compared with predicted Hb levels (417) derived from the sample hemolysis model (350).

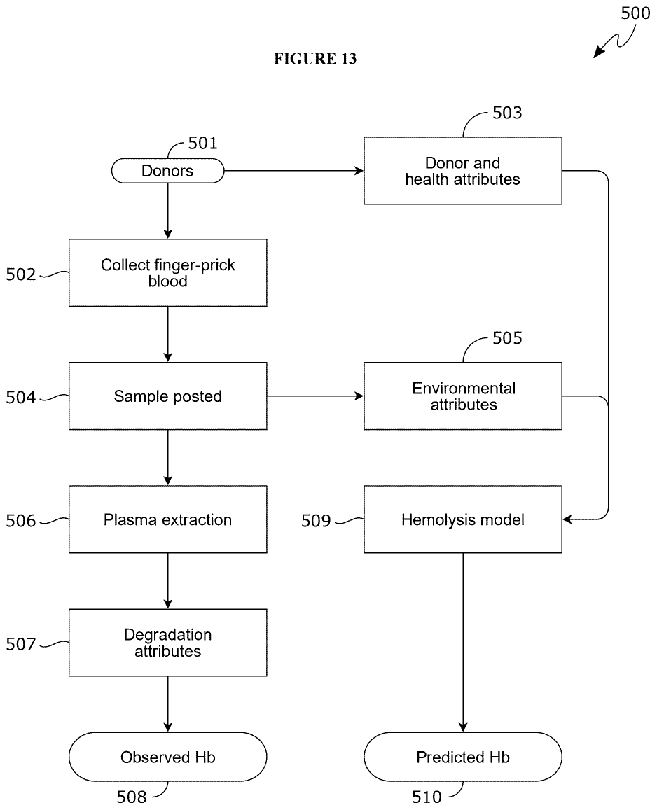

An additional real-world method (500) (FIG. 13) describes a process for predicting plasma hemoglobin (Hb) concentration in degraded samples against observed Hb values in each of the samples having been transported to the laboratory by post which was undertaken as a trial of the methods disclosed herein. The trial consisted of over 600 donors (501) that collected finger prick blood samples (502) and donor and health attributes (503) at home using the sample collection instructions provided to each donor. These samples were then sent to the laboratory for analysis by post (504) with the transit period and environmental attributes (505) captured using data loggers. Upon receipt of a sample in the laboratory, plasma was extracted from the posted blood sample (506), with degradation attributes (507), including observed Hb concentration (508) measured from the resulting plasma. Using the donor and health attributes (503) and environmental attributes (505) as inputs, the sample hemolysis model (509) was used to predict the expected level of sample Hb (510). As used in the context of FIG. 13, hemolysis and Hb are proxies for predicting degradation. Hb was quantified from plasma diluted in ddH₂O (1:10) using the Harboe method, where absorbance at 380 nm, 415 nm, and 450 nm was measured and converted to g/L using the following equation: 83.6×(2×Abs415−Abs380−Abs450).

Using the training set illustrated in FIG. 12A, the Applicant trained a model to predict sample degradation using known and controlled laboratory conditions. This model is used to make the quality assessment of samples collected at-home. The data generated in FIG. 12B create a series of data (unblinded) that simulate common mistakes made by patients that should trigger a failure of the quality assessment. The data generated by the method of FIG. 13 are from over 600 real-world examples of sample collection where the exact circumstances of sample collection and storage are unknown (blinded). To perform a quality assessment, donor, and health information in conjunction with the environmental conditions as recorded by the data logger to predict plasma Hb concentration. This is the expected value given the information known about the donor and the conditions of sample transit. The plasma concentration is then enumerated from the sample to obtain the observed sample degradation. If the observed sample degradation is greater than two times the sample degradation model error, the sample fails the quality assessment and is excluded from the analysis. It is excluded because there is a significant discrepancy between the information recorded by the data logger or by proxy and observed sample degradation, suggesting that the results may be inaccurate.

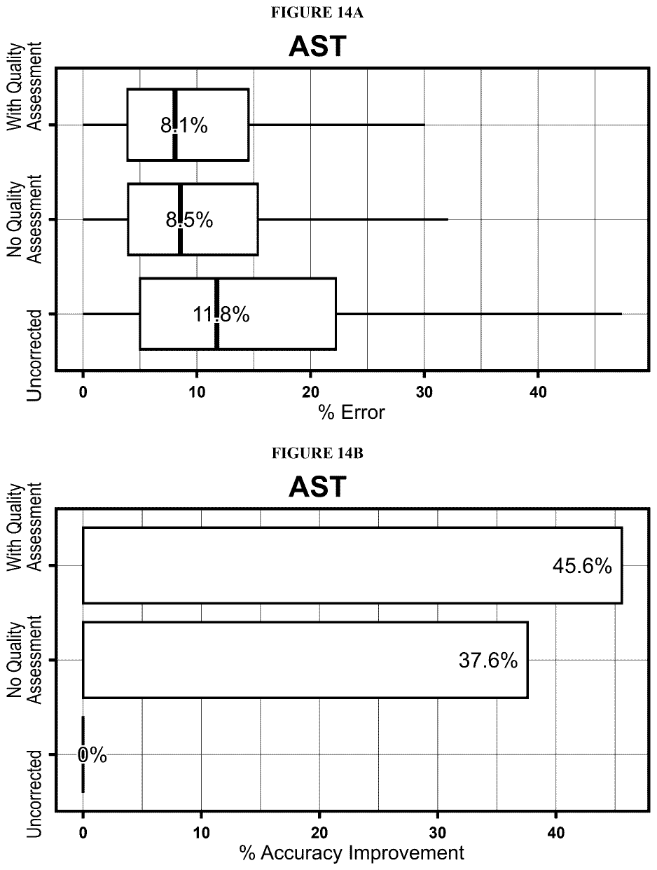

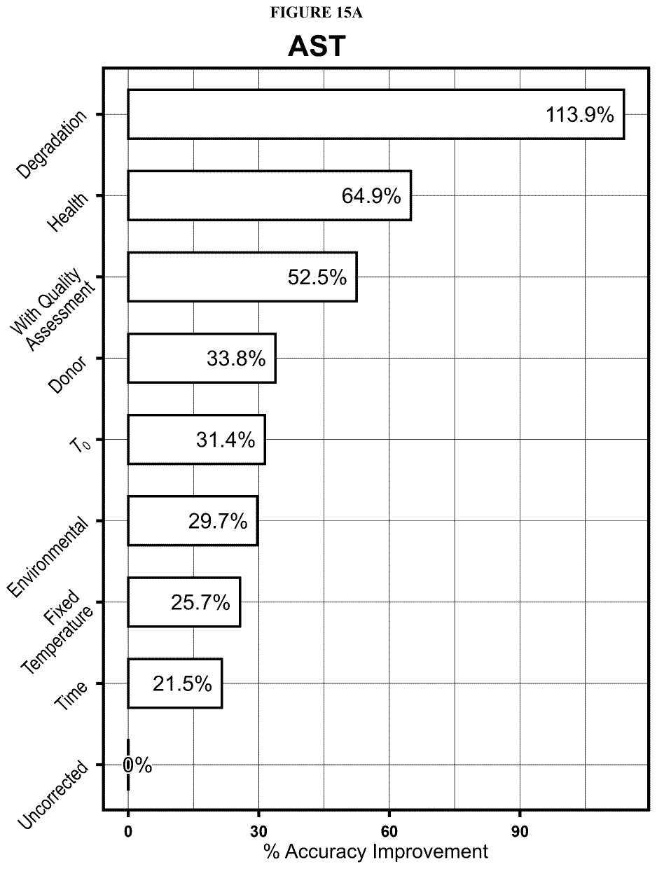

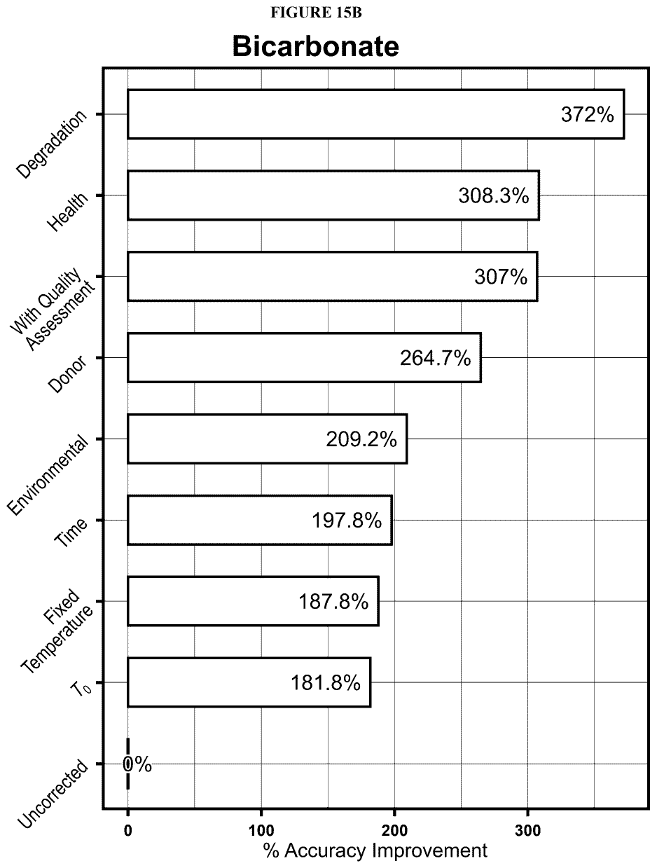

For example, application of an assessment where the expected sample quality is contrasted against the observed sample quality can help to improve quantification accuracy when the samples are transported in either controlled, or as in the real world, uncontrolled environments. Applicant tested eighteen analytes and 44% showed an improvement of test accuracy when performing a qualitative sample rejection assessment of sample quality prior to a quantitative correction of test results. There is an absolute change in % error of 3.7% between the uncorrected and quality assessment (FIG. 14A) but this provides for a % accuracy improvement of 45.6% between the uncorrected and quality assessment (FIG. 14B). The average increase in test accuracy for analytes that showed an improvement by the addition of a qualitative assessment was approximately 8% relative to the NoQualityAssessment model (FIG. 14B).

In order to test outlier detection and QC failure of the excessively hemolyzed samples, whole blood samples were created experimentally that mirror common sample collection and storage mistakes made by patients. These included exposing the samples to sample degrading conditions: (i) blood freeze and thaw; (ii) scraping blood from the finger into a sample collection tube; (iii) incomplete/ineffective blood mixing with the anticoagulant; and (iv) shaking or applying significant agitation to the blood. Samples were subjected to one of conditions (i)-(iv) in addition to a two-day dynamic temperature cycle (H4.2, min 15° C., max 28° C., average 21.7° C.). Plasma extracted from blood samples subjected to common sample collection and storage issues, which yielded a spectrum of coloration, which could be detected for using the sample degradation model for frozen samples and blood scraped from the finger during collection (refer to Table 3). Whilst samples that had been shaken and coagulated had a higher level of plasma Hb concentration relative to the reference, these were not significant. It is however hypothesized that including analytes that correlate with the degree to which a sample has been shaken or coagulated could further improve the quality assessment for at-home collected samples described herein.

TABLE 3
DETECTION OF SIMULATED OF COMMON SAMPLE
COLLECTION AND STORAGE ERRORS

| | Actual | Predicted | | |
|---|---|---|---|---|
| Condition | Hb (g/L) | Hb (g/L) | Z Score | Significance |
| Reference | 11.70 | 49.86 | −0.56 | N/A |
| Frozen | 3813.83 | 49.86 | 55.33 | *** |
| Shaken | 17.56 | 49.86 | −0.47 | NS |
| Scraped | 556.78 | 49.86 | 7.45 | *** |
| Coagulated | 26.75 | 49.86 | −0.34 | NS |

The tests and simulations described above demonstrate that the data and models developed for providing correction to biological sample measurements which may have degraded due to delays between collection and analysis, can also be used extremely effectively as a quality control screening process to predict the level of sample degradation given the information supplied by the data loggers to determine if sample degradation is in line with expectations, identify common mistake made by patient collecting samples, and to reject significantly degraded samples prior to proceeding to analyte measurements.

Of course, it is acknowledged by the Applicant and stated in section “Sample and Degradation Attributes” in above that similar performance improvements by use of quality assessment could be achieved using proxies of plasma Hb concentration, including, direct, indirect, and predicted measures of factors that are absent or in low abundance in an undegraded sample, acting as an internal clock and correlate with the degree of sample degradation. For example, a simple colorimetry assessment may be utilized as a proxy for plasma Hb concentration. Applicant found that the color of the sample could be easily determined by a machine-based colorimetry solution and accurately predict Hb concentration. This can then be used as a datapoint related to the degradation in addition to or as a proxy or replacement for another metric.

### Incremental Improvement Based on Novel and Unexpected Findings

For measurement of particular analytes in a degraded blood sample, the appropriate stability model requires different variables (i.e., in relation to particular environmental, degradation, donor and health attributes). For the minimal performance of a stability model as described herein for each of the following attributes, the model preferably comprises variables as provided in Table 4, below. For full names of test analytes abbreviated in Table 4, see Table 2 above.

TABLE 4
MINIMUM REQUIRED VARIABLE FOR
ANALYTE MEASUREMENT CORRECTION

| | Minimum |
|---|---|
| Test Analyte | Variables |
| ba-percent, eo-percent, ly-percent, mchc, | Env |
| mcv, mo-count, mo-percent, mpv, rdwsd, | |
| active_peptide_yy, adipsin, alt, bdnf, | |
| bicarbonate, ca125, ca15-3, cea, cholesterol, | |
| cortisol, creatinine, estradiol, ggt, | |
| ghrelin_total, glp1_active, glucose, hdl, hgf, | |
| ifng, il17a, il1b, il1ra, il4, il6, insulin, ldl, | |
| mcp1, opn, pancreatic_polypeptide_f, protein- | |
| total, psa-total, shbg, t4, testosterone, tnfa, | |
| triglycerides, urea, albumin, hba1c | |
| ne-percent, alp, ast, bilirubin-direct, | Env\|Deg |
| bilirubin-total, il6ra, st2 | |
| ba-count, hct, hgb, ly-count, mch, ne-count, | Env\|Don |
| plt, rbc, wbc, apo-a1, ca19-9, calcium, | |
| cpeptide, crp, eotaxin3, glucagon, icam1, il10, | |
| leptin, ngal, pai1, progesterone, t3 | |
| afp, chemerin, fgf23, gdf15, il2ra, kim1, rankl | Env\|Don\|Hea |
| eo-count, rdw | Env\|Hea |

Table 3 includes the most important model attributes for eighty-seven common test analytes required to achieve a performance improvement above an uncorrected test result. When multiple attributes are listed, it is possible to see a performance improvement with any of the listed attributes as a starting point, i.e., “Env|Don” indicates a minimum viable model may use only environmental (Env) or only donor (Don) attributes. “Env|Don” does not mean that both (Env and Don) are required to form a minimal viable model. In this way, a model can use the minimal inputs to create a viable model, based on the available data for a given dataset.

The minimum attributes for the sample degradation model for quality control purposes as discussed above in relation to quality control sampling of biological samples includes at least one of an environmental attribute, and/or a time- and temperature-dependent variable.

For maximum performance of both the stability and the quality control sampling model preferably includes the addition of any or all variables and techniques outlined herein. However, that is readily available for a given sample using existing clinical processes is optimal given the potential ease of integration. Providing corrected analyte measurements for biological samples resulting from patient-led, decentralized collection has maximum performance when a combination of stability models and sample degradation models for quality control are used in combination.

Taking all of the various variables together, Applicant can create a data set to train a model to modify a quantified value of an analyte, and dramatically, and unexpectedly improve results of this quantified value. This provides a tremendous improvement over the prior art, which, before Applicant's models, only determined the quality of a sample. Such prior models are highly flawed and essentially prevent the transmission of blood samples via the mail, without significant effort to separate the plasma and red blood cells and/or to further freeze or control temperature in some manner. Such procedures are impractical for home sampling for complexity and for cost, and as such are not commercially viable. However, the need for home testing, which opens up the opportunity for many patients to quickly and easily provide desired samples, is undeniable, and Applicant has provided herein, a technical solution to this problem.

FIGS. 15A and 15B provide an example of the model improvements when addressing certain of the parameters of the model. Indeed, what is evident from all prior figures, is that certain analytes are more sensitive to improvements by the addition of different variables than others. Indeed, the use of a data training set and creation of a stability model for quantification is precisely created to account for such differences. Therefore, when considering the given analyte, the training set can use one, two, three or more of the assessments to improve the accuracy of the quantified result (see Table 3, which provided minimum needed for each analyte).

In certain instances, a given analyte may only require a modest improvement in accuracy in order to ensure it is relevant. For example, when considering a particular analyte, it may be necessary to only detect whether the analyte is present above a given value, as the range of acceptable values is large. In such an example, the given acceptable error rate for determining a yes or no answer as to whether that analyte is acceptable at the given quantified result may be very large. And as such, accounting for only the time in transit and temperature, for example, may be sufficient. In others, accounting for time and temperature and another variable may be acceptable. For others, the inclusion of a donor attribute, alone or with time or time and temperature may provide the necessary improvement. In essence, the level of accuracy required for a given test is dependent on the intended use, and a suboptimal model may meet this lower requirement.

For those samples, by contrast, wherein the analyte requires a much lower acceptable error rate, such as for diagnosis of a disease, a much higher level of scrutiny should then be applied to the model. As is evident in FIGS. 15A and 15B by inclusion of the multiple improvements to the model, certain analytes can be increased in their accuracy by 113.9% and 37.2%, respectively. However, in other data not shown in the figures, analyte accuracy has increased by 600% or more. When faced with an analyte that requires such scrutiny, a tighter model, and a data training set sufficient to train that model is absolutely necessary. Each of these FIGS. 15A and 15B show that by inclusion of stepwise elements to the model, the accuracy compared to the true value can be dramatically improved.

### Development of the Stability and Quality Assessment Models

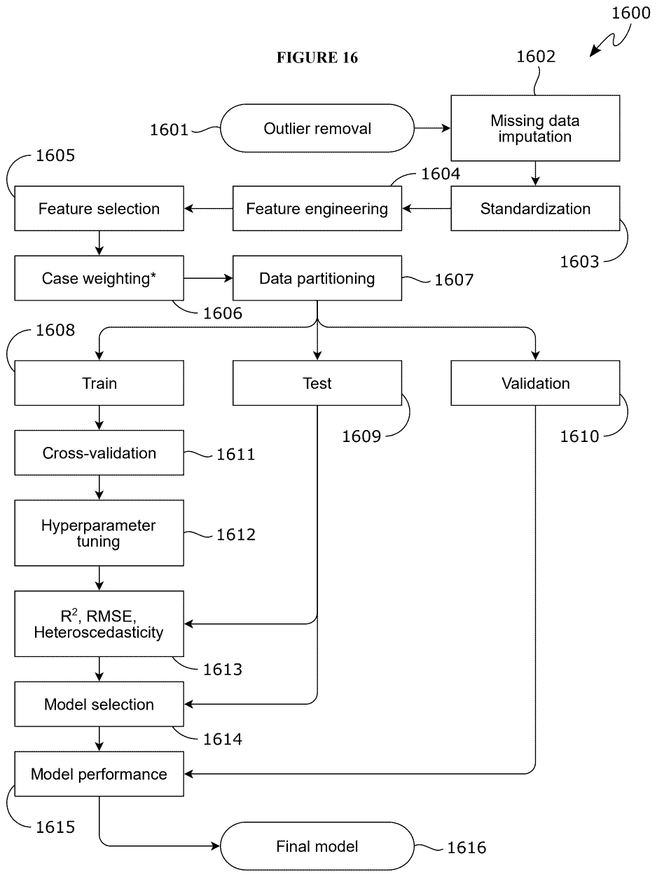

Each of the data training sets is appropriately created by the various methodologies detailed herein. Upon capture of the entire data training set, the model may be executed by systems of the present disclosure. The systems include the use of one or more computers, which are known to those of ordinary skill in the art, and which are suitable to run and execute software to perform the modifications to the quantified values of desired analytes. An example of training the model is provided by FIG. 16.

Therefore, a model can be trained on the data sets, as outlined herein. The model ultimately can use a variety of parametric, nonparametric, AI, or other modeling to best fit the data based on the dataset. The data sets generated in FIGS. 1, 2, 12A, 12B, and 13 take a similar structural form, consisting of environmental, donor, health, and degradation variables (columns). Each row of the data set represents a sample subjected to a different environmental condition (FIG. 1 step [106], FIG. 2 step [206], FIG. 12A step [306], FIG. 12B step [407], and FIG. 13 step [504]). Environmental variables can be obtained from a data logger or other proxy for environmental conditions. Donor and health variables or proxies for these are obtained from the participant donating the blood. Sample and degradation variables or proxies for these are determined directly or indirectly from the sample itself. Alternatively, degradation variables can be generated by prediction from a separate model using one or more of environmental and/or donor and health variables (model stacking). This raw data set must undergo a series of preprocessing steps (1601-1606) in preparation for training a model (1607-1609) and assessment of model performance (1610-1613). This process outlined in FIG. 16 is largely the same process for creation of stability and degradation models; however, the inclusion of each step should be considered by a person skilled in the art. The process (1600) depicted in FIG. 16 is what was consistently found to generate optimal model performance, although a similar performance can be obtained by the omission of particular steps.

Outliers were identified for each variable depending on the underlying data distribution, using parametric and nonparametric methods. Handling of outliers (1601) was assessed on a case-by-case basis with remedies including removal for measurement or data entry errors or transformation and bootstrapping for outliers that were the result of natural population variances. Reasons for missing data were investigated to categorize missing data under missing completely at random (MCAR), missing at random (MAR) and missing not at random (MNAR), and to assess association with outcome. Multiple imputation (1602) was used to address MCAR and MAR, with complete case analysis used for instances of MNAR to avoid bias in regression coefficients. The data transformation, standardization, and/or normalization (1603) techniques were chosen for each variable using Q-Q plots and the Shapiro-Wilk Test or Kolmogorov-Smirnov-Test of normality. Feature engineering (1604) encompassed hot-encoding categorical variables with multiple outcomes. Variables with near zero variance or with a substantial proportion of missing data were removed, and variable selection by importance determined by a variety of methods appropriate for the model library formed feature selection (1605) preprocessing step. Depending on the intended use case of the model, case weighting (1606) involved assigning different levels of importance or influence to individual data points. This was performed in cases where data was (i) suspected of being unreliable, e.g., as a result of imputation (1602), or (ii) important to biasing performance to a use case. Case weighting for posted samples were weighted using a Poisson and exponential distributions that approximate sample transit duration, i.e., the probability of a sample being delivered within a timeframe.

For correction of test results, the test value for the desired analyte is supplied with one or many environmental, donor, degradation, and health attributes for a given sample. The degradation model predicts the expected level of degradation and contrasts with the observed sample degradation. If the expected degradation is within a margin of error of the observed degradation, the sample passes the quality assessment. The stability model for the desired analyte is subsequently used to predict a relative change known as the correction coefficient. The test value is converted to the corrected value, by dividing the test value by the correction coefficient. The corrected value approximates the true value of a performance described by the invention herein.

Processed data was partitioned (1607) into training (1608), testing (1609) and validation (1610) sets. Training (1608) and testing (1609) sets, consisting of an 80:20 split, respectively, were derived from data generated in vitro under laboratory conditions (FIGS. 1, 12A and 12B), whereas the validation data set consisted of real-world data from posted samples (FIGS. 2 and 13). Environmental, donor, health, and degradation attributes were used as the dependent variables. The independent variable was the relative difference between the true value and test value (test value/true value). To avoid overfitting, ten-fold cross-validation was performed (1611), where the training set is further divided into k equal-sized subsets. The model was trained on k−1 folds and tested on the remaining fold, with this process repeated k times such that each fold. Hyperparameters are parameters that are set before the training process begins and cannot be directly learned from the data. Tuning of model-specific hyperparameters (1612) was performed using a grid search that creates a matrix of all possible combinations of incremental changes between a prespecified range of values. Model accuracy was determined using the test dataset, comparing predicted relative difference (model output) to observed relative difference. Accuracy metrics used included R², residual mean squared error (RMSE), and heteroscedasticity that assess fit, error, and residual variance, respectively (1613). The best performing model was selected (1614) using a weighted rank based on a model maximizing R² and minimizing RMSE and heteroscedasticity. Model performance (1615) was assessed using the independently collected validation data set to create the final model (1616).

In all cases, the models may be individual models, the models may be a plurality of models, e.g., a system of models, competing models, or an ensemble of models that work together. The models may be generic to certain factors or may be precise to other factors. In all cases, the models herein are unique in that they utilize both known information and in many cases information from the sample itself to be inputs as to elements of the sample. Thus, where a donor sample provides into the model the donor attributes, elemental attributes, and then includes degradation attributes from the sample itself, the models are able to dramatically increase the accuracy and precision for calculating analytes from a sample that is otherwise degraded based on prior modeling systems. This technical solution and the methods herein that employ this technical solution provide for quantified levels within a sample and dramatically reduce the number of samples that would otherwise be rejected.

### Obtaining a Remote Sample and Quantifying Results

The collection kit and required tests can be ordered independently by the patient or by a general practitioner (“GP”) after an in-person, telehealth consultation or provided as part of a screening program and delivered to the patient's home by registered mail. The collection kit is a listed medical device that contains all of the consumables and instructions necessary for a patient or caregiver to collect a capillary blood sample. The patient or caregiver labels the sample with the patient's name, date of birth and time of collection, packages and returns the sample by mail to a partnered pathology laboratory and analyzes using standard pathology analyzers.

In certain embodiments, the return package contains an active sensor that is constantly recording the environmental condition during the sample transit. Upon receipt of the sample by the laboratory, the sensor data is securely uploaded to the cloud automatically using a geofence and radio-frequency identification (RFID) or near-field communication (NFC). If the return package does not utilize an active sensor, the environmental conditions are obtained by proxy, by capturing the environmental temperatures manually or via an API or via another accessible database. In this way, the conditions of the sample are known based on the dynamic nature of the environmental conditions to which the sample is subjected. The blood sample is analyzed on standard pathology analyzers. The laboratory requests correction of test value by stability model by API or using the software integrated with the laboratory equipment or laboratory information management system. Applicant takes the test value(s) and provide the corrected value(s), which are returned to the laboratories information management system using standardized transfer for clinical and administrative health data and onwards to the referring doctor for a follow up consultation.

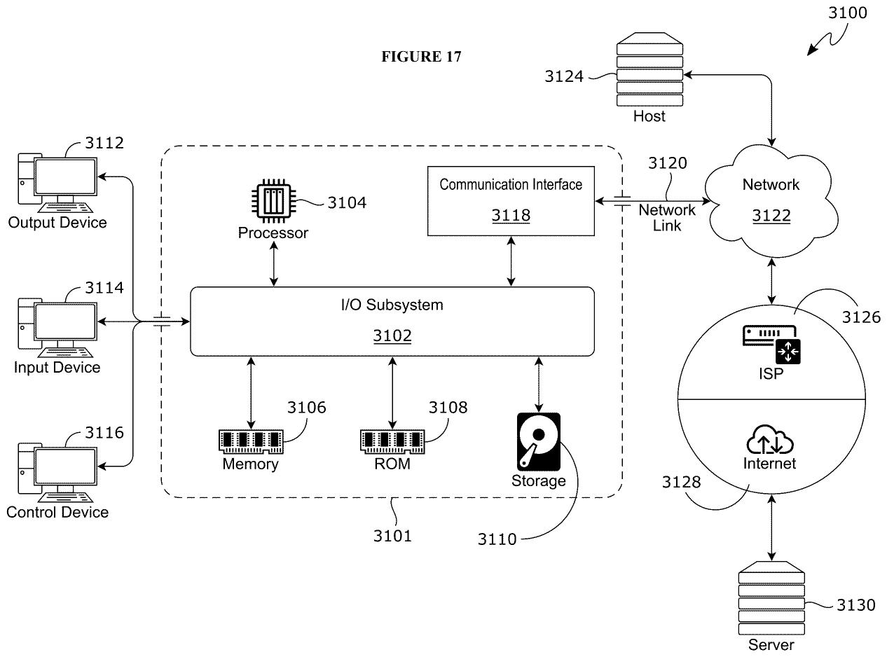

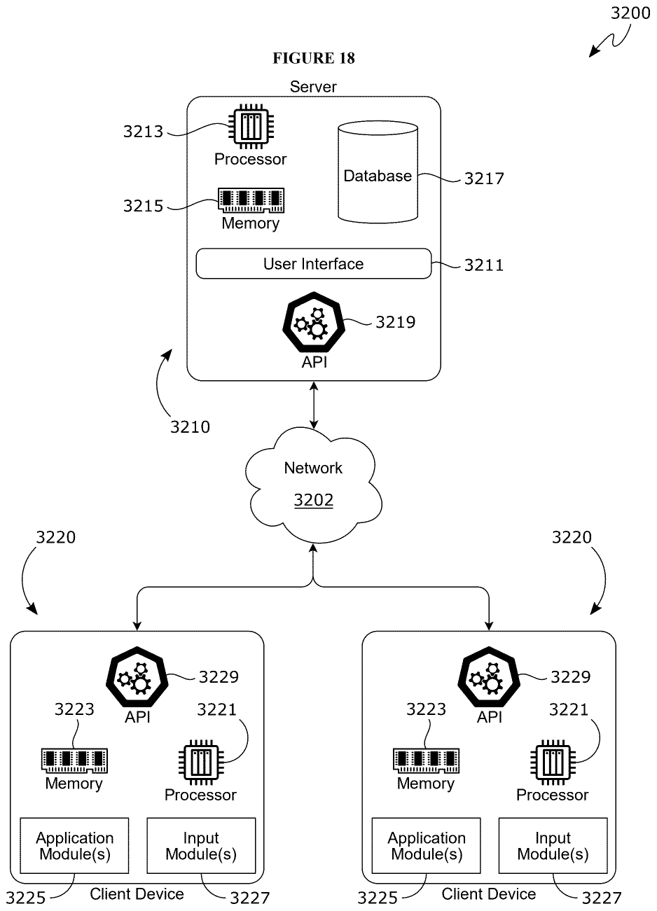

FIG. 17 is a block diagram that illustrates an example computer system with which an embodiment of the methods described herein may be implemented, for example, methods (100, 200, 300, 400 and 500) or methods as disclosed in the above description including: generating a training set for a machine learning model; creating a machine learning model based on the difference between test values and true values of analyte measurements of a degraded biological sample; or correcting test analyte values using a machine learning module.

The system and methodologies utilized herein are conceived to be able to be processed by a computer in a local setting or, most preferably via a cloud setting. In the example implementation of FIG. 17, a computer system (3100) and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

Computer system (3100) includes an input/output (I/O) subsystem (3102) which may include a bus and/or other communication mechanism(s) for communicating information and/or instructions between the components of the computer system (3101) over electronic signal paths. At least one hardware processor (3104) is coupled to I/O subsystem (3102) for processing information and instructions. Hardware processor (3104) may include, for example, a general-purpose microprocessor or microcontroller and/or a special-purpose microprocessor such as an embedded system or a graphics processing unit (GPU) or a digital signal processor or ARM processor. Processor (3104) may comprise an integrated arithmetic logic unit (ALU) or may be coupled to a separate ALU.

Computer system (3100) includes one or more units of memory (3106), for electronically digitally storing data and instructions to be executed by processor (3104). Memory (3106) may include volatile memory such as various forms of random-access memory (RAM) or other dynamic storage device. Memory (3106) also may be used for storing temporary variables or other intermediate information during execution of instructions to be executed by processor (3104).

Computer system (3100) further includes nonvolatile memory such as read only memory (ROM) (3108) or other static storage device for storing information and instructions for processor (3104). Storage (3110) is an example of a nontransitory computer-readable medium that may be used to store instructions and data which, when executed by the processor (3104), causes performing computer-implemented methods to execute the techniques herein.

The instructions in memory (3106), ROM (3108) or storage (3110) may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming, or other functions; data protocol instructions or stacks to implement TCP/IP, HTTP, or other communication protocols. The instructions may implement a Web server, Web application server, or Web client. The instructions may be organized as a presentation layer, application layer and data storage layer such as a relational database system using structured query language (SQL) or no SQL, an object store, a graph database, a flat file system or other data storage.

Computer system (3100) may be coupled to at least one output device (3112), such as a display or other output device.

At least one input device (3114) is coupled to I/O subsystem (3102) for communicating signals, data, command selections or gestures to processor (3104). Such devices may include but are not limited to keyboards, touchscreens, buttons, sensors, global positioning system (GPS), radio frequency (RF) infrared (IR) and the like, as are understood by those of ordinary skill in the art. Another type of input device is a control device (3116), which may perform cursor control or other automated control functions such as navigation in a graphical interface on a display screen, alternatively or in addition to input functions. An input device (3114) may include a combination of multiple different input devices, such as a video camera and a depth sensor.

In another embodiment, computer system (3100) may comprise an Internet of things (IOT) device in which one or more of the output device (3112), input device (3114), and control device (3116) are omitted, or in such an embodiment, the input device (3114) may comprise one or more cameras, motion detectors, thermometers, microphones, seismic detectors, other sensors or detectors, measurement devices or encoders and the output device (3112) may comprise a special-purpose display such as a single-line LED or LCD display, one or more indicators, a display panel, a meter, a valve, a solenoid, an actuator or a servo.

Output device (3112) may include hardware, software, firmware, and interfaces for generating position reporting packets, notifications, pulse or heartbeat signals, or other recurring data transmissions that specify a position of the computer system (3100), alone or in combination with other application-specific data, directed toward host (3124) or server (3130).

According to one embodiment, the techniques herein are performed by computer system (3100) in response to processor (3104) executing at least one sequence of at least one instruction contained in main memory (3106). Such instructions may be read into main memory (3106) from another storage medium, such as storage (3110). Execution of the sequences of instructions contained in main memory (3106) causes processor (3104) to perform the process steps described herein. In alternative embodiments, hardwired circuitry may be used in place of or in combination with software instructions.

Computer system (3100) also includes a communication interface (3118) coupled to bus (3102). Communication interface (3118) provides a two-way data communication coupling to network link(s) (3120) that are directly or indirectly connected to at least one communication network, such as a network (3122) or a public or private cloud on the Internet. For example, communication interface (3118) enables communication between one or more computing systems. Network (3122) broadly represents a local area network (LAN), wide area network (WAN), campus network, internetwork, or any combination thereof. In any such implementation, the communication interface (3118) sends and receives electrical, electromagnetic, or optical signals over signal paths that carry digital data streams representing various types of information.

Network link (3120) typically provides electrical, electromagnetic, or optical data communication directly or through at least one network to other data devices, using, for example, satellite, cellular, WiFi, or Bluetooth technology. For example, network link (3120) may provide a connection through a network (3122) to a host computer (3124).

Furthermore, network link (3120) may provide a connection through network (3122) or to other computing devices via internetworking devices and/or computers that are operated by an Internet service provider (ISP) (3126). The ISP (3126) provides data communication services through a worldwide packet data communication network represented as Internet (3128). A server computer (3130) may be coupled to the Internet (3128). The server (3130) broadly represents any computer, data center, virtual machine, or virtual computing instance with or without a hypervisor, or computer executing a containerized program system such as DOCKER or KUBERNETES.

Computer system (3100) can send messages and receive data and instructions, including program code, through the network(s), network link (3120) and communication interface (3118). In the Internet example, a server (3130) might transmit a requested code for an application program through the Internet (3128), ISP (3126), local network (3122), and communication interface (3118). The received code may be executed by processor (3104) as it is received, and/or stored in storage (3110), or other nonvolatile storage for later execution.

In a particular arrangement of a SaaS application comprising a cloud-based architecture (3200), an example of which is provided in FIG. 18, SaaS system (3200) comprises a client-accessible server application system (3210) for providing the SaaS application, the server (3210) comprising: (i) a user interface (3211) providing responsive dashboards for data presentation and user interaction by third-party client devices (3220), (ii) arithmetic logic unit or processor(s) (3213) for performing computer program code instructions. The processor(s) (3213) may be a reduced instruction set computer (RISC) or complex instruction set computer (CISC) processor or the like, (iii) memory (3215) for storage of computer code instructions, for example as disclosed in any one of the aspects or arrangements of the correction models discussed above, (iv) database (3217) for storage of the stability consensus functions and models as discussed above for correction of analyte measurements of degraded biological samples and cloud storage of aggregated stability consensus model data including correction coefficients for one or more analytes, (v) server application programming interface(s) (API[s]) (3219) encapsulating portal functionality, using open standards to facilitate third-party vendors in building platform-compatible apps and connectors built on open-standard APIs to facilitate connection to third-party client devices (3220), and (vi) wherein user interface (3211) is configured to provide corrected output data comprising the corrected analyte test result of the biological sample to the third party client requestor (3220).

Processor(s) (3213), memory (3215), and database (3217) preferably are configured to provide computer software code instructions and data for implementation of: (i) a data validation engine which is configured to receive test values and corresponding degradation attribute data from a third-party client such as a measurement laboratory, (ii) optionally, a quality control module configured to screen the received sample measurements and supplied degradation attribute information supplied by client to determine if sample degradation is in line with expectations, and to reject significantly degraded samples prior to proceeding to analyte measurements, and (iii) correction module configured for correcting test values of a degraded biological sample having undergone changes on the basis of environmental factors experienced by the sample.

Client devices (3220) of a client requestor, for example a biological test laboratory, comprise: (i) processor(s) (3221) for execution of software code instructions stored in memory (3223) for interacting with application module(s) (3225) and for interacting with server (3210) via client API(s) (3229) over a network (3202), such as, for example, the Internet or similar communications network, and (ii) application module(s) (3225) configured to receive biological sample test data and corresponding degradation attributes via input module(s) (3227).

The correction data received from client device(s) (3220) is performed using a correction coefficient based on a machine learning model trained on a dataset of degraded samples to provide a database of models/equation that take environmental, donor, and degradation attributes as inputs and which output the correction coefficient with respect to a plurality of testable analytes of the biological sample. The correction module of server (3210) is configured to receive analyte test results of a degraded biological sample, apply the correction factor applicable to an analyte to provide a corrected test result.

The SaaS system (3200) is designed for scalability and extensibility to allow customization to accommodate the needs of individual healthcare organizations. Examples of such customization include, among other uses as would be appreciated by the skilled addressee: (i) Customer A needs a solution that automatically links the output of the CDS to the input of the predictive analytics (PA). A module with a prestored set of queries to be automatically run on the PA system for each of a particular set of patients; (ii) Customer B wishes to automatically aggregate clinical data from certain patients as part of a planned research study; (iii) Customer C plans to deploy biometric monitoring for select patients and is interested in setting the CDS diagnostic report for autorun to generate reports on these patients when certain events are registered in real time from the sensors.

In use, of the SaaS system, the biological testing laboratory clients receives biological samples and associated data with respect to one or more degradation attributes experienced by the received biological samples; conducts testing of the samples of one or more analytes to record a test value for the analyte; uploads sample test value data and to the software application so that a correction calculation could be made. The software application would apply the relevant biomarker model to the uploaded biomarker result and provide back a “corrected” value along with the original value, the correction applied, and the version of the model that was applied to the result.

### CONCLUSION

In conclusion, remote sample collection is vital to addressing the increasing health disparity for patients living in rural and remote communities that cannot easily access common pathology services like blood testing. However, strict quality control measures implemented by medical pathology laboratories reject most samples collected in an at-home setting due to the inherent time delay and degradation that occurs during sample transit that would, without a solution, cause issues with accuracy of test results. Prior art solutions have proven to be prohibitively expensive, enough so that remote sample collection is not commonplace in clinical practice. The invention described by the Applicant herein, is a cost-effective, software-based solution that (i) expands the catalog to include labile tests not previously available in the at-home setting by extending the sample viability window, (ii) improves testing precision and accuracy for nonlabile analytes and sample transported in controlled and uncontrolled environments, and (iii) creates a new quality control standard for samples collected at-home leading to lower sample recollection rates by factoring in each sample transit condition into the assessment. The embodiment detailed herein will revolutionize how healthcare can be delivered at-home and on the patient's terms.

What is claimed is:

1. A method for generating a training set for correction of analyte test results in a biological sample, the method comprising:
a. obtaining a biological sample from a plurality of donors wherein donor attribute data is collected at the time of collecting the biological sample to obtain a plurality of biological samples and associated donor attributes;
b. dividing the collected biological sample into a plurality of aliquots and dividing the aliquots into a first subset of aliquots and a second subset of aliquots;
c. measuring one or more analytes of the biological sample from the plurality of aliquots of the first subset of aliquots to provide a true value of one or more analyte characteristics for each aliquot in the first subset of aliquots;
d. subjecting each of the plurality of aliquots of the second subset of aliquots to an environmental attribute and capturing the environmental attribute by measurement or a proxy to obtain a plurality of degraded aliquot samples;
e. for each degraded aliquot sample, measuring one or more analytes of the biological sample to provide a test value of the one or more analyte characteristics;
f. comparing the true values and test values for each of the sample analytes to determine a difference between the true values and respective test values; and
g. generating a training set for training a machine learning or artificial intelligence system, the training set based on the relative difference between the true values and the test values corresponding to the environmental attribute and/or the donor attributes.

2. The method of claim 1 further comprising obtaining a sample degradation attribute for each second aliquot.

3. The method of claim 1 wherein the donor attributes, environmental attributes, and/or degradation attributes are a proxy for such attributes.

4. The method of claim 1 further comprising quantifying a plurality of analytes of each aliquot sample to obtain a true value for each of the plurality of analytes measured.

5. The method of claim 1 further comprising wherein the second subset of aliquots is subjected to environmental conditions and capturing said environmental conditions wherein the environmental conditions are time, temperature, or both.

6. The method of claim 5 wherein the environmental conditions are dynamic environmental conditions.

7. The method of claim 1 further comprising comparing the difference in the true value and the test value to construct a machine learning or artificial intelligence model capable of predicting a correction factor in order to correct test results using the machine learning or artificial intelligence model trained on the training set.

8. The method of claim 1 wherein the machine learning or artificial intelligence model is a parametric or nonparametric machine learning model; and/or
wherein the machine learning or artificial intelligence model is constructed using a multivariate adaptive regression spline (MARS) method for each analyte of the biological sample of interest; and/or
wherein the machine learning or artificial intelligence model is calculated using either a locally weighted scatterplot smoother (LOWESS) or a polynomial fit model for each analyte of the biological sample of interest.

9. The method of claim 1 wherein the biological sample is a blood sample and the measuring comprises measuring one or more analytes from a plasma or serum fraction of the blood sample.

10. The method of claim 1 wherein the donor attributes are selected from the group consisting of: age, sex, ethnicity, BMI, fasting status, time of day, medication, medical conditions, and combinations thereof.

11. The method of claim 1 wherein the environmental attributes comprise one or more of a proxy of time, temperature, humidity, pressure, geolocation, acceleration, and light.

12. The method of claim 2 wherein degradation attributes comprise one or more of a proxy of entropy of a biological sample, red blood cell lysis, white blood cell lysis, presence or absence of DNA, sodium, chloride, potassium, an analyte different from an analyte of interest, and evaporation.

13. The method of claim 12 wherein the degradation attribute is calculated by using a first model and comprising using the calculations from the first model as inputs to a second model wherein the inputs to the second model are representative of estimated degradation of the biological sample.

14. A computer-implemented method of training a model for analyte correction comprising:
a. collecting a set of biological samples;
b. separating the set of biological samples into at least a first set of aliquots and a second set of aliquots;
c. applying one or more environmental attributes to the second set of aliquots;
d. creating a first training set comprising the first set of aliquots of biological samples, each said biological sample having a known analyte value;
e. training the model in a first stage using the first training set;
f. creating a second training set for a second stage of training comprising the second set of aliquots;
g. training the model in a second stage using the second training set; and
h. comparing the model of the first stage to the model of the second stage to define the analyte correction model.

15. The computer-implemented method of claim 14 further comprising associating a donor attribute to each of the first set of aliquots and the second set of aliquots.

16. The computer-implemented method of claim 14 further comprising associating a degradation attribute to the biological sample in each of the second set of aliquots.

17. The computer-implemented method of claim 16 wherein the environmental attribute and/or the degradation attribute is obtained by a proxy.

18. The computer-implemented method of claim 14 further comprising a third training set, said third training set defined by using the analyte correction model to predict a degradation attribute, and training a stacked model based upon the third training set.

19. A method of creating a model for analyte stability comprising:
a. collecting a set of test value measurements for a given analyte, each test value measurement having a corresponding true value measurement wherein the given analyte is obtained from a mammalian biological sample;
b. associating each test value measurement and corresponding true value measurement with a donor attribute and storing the test value measurements and true value measurements in a database;
c. associating an environmental attribute to each test value measurement to create a modified set of test values;
d. creating a first training set comprising the collected set of test values, the corresponding true values, and the modified set of test values; and
e. training a sample stability model with the first training set.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)
